(19) United States
(12) Patent Application Publication
(10) Pub. No.: US 2005/0181360 A1
(43) Pub. Date: Aug. 18, 2005

(54) Assay for cell cycle modulators based on the modulation of cyclin d1 degradation in response to ionising radiation

(76) Inventors: Rene Bernards, Amsterdam (NL); Reuven Agami, Amsterdam (NL)

Correspondence Address: NIXON & VANDERHYE, PC, 901 NORTH GLEBE ROAD, 11TH FLOOR, ARLINGTON, VA, 22203, US

(21) Appl. No.: 10/275,914

(22) PCT Filed: May 14, 2001
(86) PCT No.: PCT/GB01/02099

(30) Foreign Application Priority Data
May 12, 2000 (GB) 0011557.6
Jul. 7, 2000 (GB) 0016783.3

(51) Int. Cl. C12Q 1/68; C12N 15/85

(52) U.S. Cl. 435/6; 435/455

(57) ABSTRACT

The present invention relates to the finding that cyclin D1 is targeted for destruction in cells which have been exposed to ionising radiation (IR). This finding gives rise to an assay for modulators of cell cycle control, which assay comprises: (a) providing a cell in culture together with a potential modulator compound, said cell expressing a cyclin D1 which undergoes degradation in response to DNA damage; (b) exposing said cell to a DNA damaging agent; and (c) determining the extent to which the presence of the potential modulator compound inhibits the degradation of said cyclin D1. This finding further gives rise to an assay for modulators of cell cycle control, which assay comprises: (a) providing a cyclin D1, the APC or a compound thereof which interacts with cyclin D1, together with a potential modulator compound; and (b) determining the extent to which the presence of the potential modulator compound inhibits the interaction of said cyclin D1 and APC or component thereof. In particular where the component of the APC is a protein cdc20.

### FIELD OF THE INVENTION

The present invention relates to the finding that cyclin D1 is targeted for destruction in cells which have been exposed to ionising radiation (IR). The finding gives rise to novel targets for the control of the cell cycle and the treatment of diseases such as cancer.

### BACKGROUND TO THE INVENTION

Cyclins are essential components of the cell cycle machinery. They function to bind and activate their specific cyclin dependent kinase (CDK) partners. During progression through the G1 phase of the cell cycle two major types of cyclins are required: D-type cyclins and cyclin E. Together they cause phosphorylation of the retinoblastoma family of tumor suppressor proteins (pRb, p107, and p130) in G1 and abrogate their inhibitory activity (Lipinski and Jacks, 1999). The three D type cyclins are very similar (more than 70% identity) but share very little homology with cyclin E. The D cyclins activate primarily CDK4 and 6 whereas cyclin E activates CDK2. Furthermore, during cell cycle progression D cyclins are active at mid-G1 whereas cyclin E appears later just prior to the G1/S transition (Draetta, 1994; Sherr, 1994; Sherr and Roberts, 1995). Therefore, progression through G1 depends initially on D cyclin-CDK4/6 protein complexes and later on cyclin E-CDK2. Given the crucial part that D type cyclins play in progression through the cell cycle, it is perhaps not surprising that their expression is frequently deregulated in cancer (Sherr, 1995).

Cell cycle arrest in response to either mitogen deprivation or genotoxic stress requires CDK inhibitors (CKIs) of the CIP/KIP family which includes p21^{cip1}, p27^{kip1} and p57^{kip2} (Morgan, 1995; Sherr, 1995). Members of this family bind both CDK2 and CDK4 complexes, but act as potent inhibitors of cyclin E-CDK2 protein complexes and as positive regulators in the case of D cyclins-CDK4/6 (Sherr and Roberts, 1999). D type cyclins connect extracellular signalling pathways to the cell cycle machinery as their promoters respond to a variety of mitogenic signals, such as those transduced by the Ras and APC-β-catenin-Tcf/Lef pathways (Morin, 1999; Tetsu and McCormick, 1999). Furthermore, mitogen deprivation accelerates cyclin D1 proteolysis via the PI3K-PKB/Akt-GSK3-β pathway. GSK3-β phosphorylates cyclin D1 at threonine 286, which triggers its nuclear export, ubiquitination and degradation (Diehl et al., 1998; Diehl et al., 1997). Mitogenic signals activate the PI3K-PKB/Akt pathway, which in turn inhibit GSK3-β kinase activity and stabilize cyclin D1 protein. Expression of c-Myc also causes activation of the cyclin D1 and D2 promoters. Increased protein levels of D cyclins results in complex formation with their CDK partners, which function to sequester p21^{cip1} and p27^{kip1} away from cyclin E-CDK2 complexes, allowing G1-S progression (Bouchard et al., 1999; Perez-Roger et al., 1999).

DNA damage checkpoints control the timing of cell cycle progression in response to genotoxic stress (reviewed in (Weinert, 1998)). Arrest in G1 is thought to prevent aberrant replication of damaged DNA and arrest in G2 allows cells to avoid segregation of defective chromosomes. Primary among mammalian checkpoint genes is the tumor suppressor p53. In response to DNA damage, such as IR, p53 is required for G1 arrest (Kastan et al., 1991; Kastan et al., 1992; Kuerbitz et al., 1992; Livingstone et al., 1992; Yin et al., 1992), apoptosis (last reviewed in Sionov and Haupt, 1999) and to sustain arrest of cells prior to M phase (Bunz et al., 1998; Chan et al., 1999). In response to IR, rapid phosphorylation of p53 by the ATM-CHK2 pathway on serines 15 and 20, leads to release of Mdm2 and stabilization of p53 (Meek, 1999 and references therein).

Since p53 acts primarily as a transcription factor, stabilization of p53 activates transcription of target genes required for various aspects of the genotoxic stress response. In particular, p53 transactivation is required to induce an efficient G1 arrest (el-Deiry et al., 1993; Waldman et al., 1995). An essential transcriptional target of p53 in induction of G1 arrest is p21^{cip1} (Waldman et al., 1995). Accumulation of p21^{cip1} inhibits cyclin-E/CDK2 activity and therefore G1-S transition. However, as this p53 response depends on transcriptional activation, the time required to execute this type of cell cycle arrest is rather long and exceeds in most cases eight hours.

### DISCLOSURE OF THE INVENTION

We have now found that cells initiate a fast and efficient, p53-independent, G1 arrest after DNA damage caused by IR. We have identified a p53-independent mechanism that implements an efficient G1 arrest immediately after exposure to genotoxic stress. In particular, we have found that IR, an inducer of DNA damage, induces a rapid degradation of cyclin D1 in cells, and that this inhibits progression of cells through the G1 phase of the cell cycle. Degradation of cyclin D1 is mediated through a motif “RXXL” found in the N-terminal region of cyclin D1.

We have also found that in tumour cells which express cyclin D1 appear to retain this rapid response. This finding has potential relevance in the treatment of cancer by irradiation, where problems may be encountered in overcoming the resistance of cells to irradiation. Because irradiation induces a G1 arrest in tumour cells, this may provide the cells with an opportunity to initiate DNA repair prior to replication, thus ensuring survival of the tumour. By blocking this protective mechanism, the efficacy of therapy in which DNA damage is induced in target cells may be enhanced.

Accordingly, the present invention provides an assay for a modulator of cell cycle control, which assay comprises:
(a) providing a cell in culture together with a potential modulator compound, said cell expressing a cyclin D1 which is undergoes degradation in response to DNA damage;
(b) exposing said cell to a DNA damaging agent; and
(c) determining the extent to which the presence of the potential modulator compound inhibits the degradation of said cyclin D1.

The potential modulator compound may be a cellular protein, which can be introduced into the cell by providing for its expression from a cDNA. Accordingly, another aspect of the invention provides a method to discover genes whose protein products participate in the same signalling pathways as cyclin D1 degradation. Thus the invention provides an assay which comprises:
(a) providing a cell in culture, said cell expressing a cyclin D1 which undergoes degradation in response to DNA damage;
(b) introducing into said cell a member of a cDNA library operably linked to a promoter which expresses said cDNA in said cell;
(c) exposing such cell to a DNA-damaging agent and determining the extent to which the expression of said cDNA modulates the degradation of said cyclin D1; and optionally
(d) isolating said cDNA.

In a further aspect, we have found that the “RXXL” motif, when transplanted to a different protein (in the examples below, cyclin D2), acts as a destruction box which directs the protein for degradation in response to IR. Thus in a further embodiment of the invention, there is provided an assay which comprises:
(a) providing a cell in culture together with a potential modulator compound, said cell expressing a reporter protein having an RXXL destruction box and which protein undergoes degradation in response DNA damage;
(b) exposing said cell to a DNA damaging agent; and
(c) determining the extent to which the presence of the potential modulator compound inhibits the degradation of said reporter protein.

In another aspect, our experiments suggest that the cyclin D1-derived RXXL motif targets cyclin D1 (or a protein comprising this motif) to the anaphase promoting complex (APC) of a cell. The APC is a complex of about a dozen proteins which regulate various aspects of the cell cycle. While not wishing to be bound by any one particular theory, it is believed that the APC marks cyclin D1 for proteolysis. The interaction between the APC and the cyclin D1 provides a further target for therapeutic intervention. Thus in this aspect, the invention provides an assay for a modulator of the cell cycle which assay comprises:
(a) providing a cyclin D1, the APC or a component thereof which interacts with cyclin D1, together with a potential modulator compound; and
(b) determining the extent to which the presence of the potential modulator compound inhibits the interaction of said cyclin D1 and APC or component thereof.

The data provided herein indicate that the interaction between cyclin D1 and the APC may be mediated by CDK4. Thus in the abovementioned aspect of the invention, the assay may be performed in the presence of a CDK4.

Our experiments suggest that in the interaction between cyclin D1 and the APC, the protein to which cyclin D1 binds is Cdc20, an activator of the APC. It is believed that Cdc20 is a crucial component for the degradation of cyclin D1 in response to DNA damage, by this pathway. Thus the abovementioned aspect of the invention further provides an assay wherein the component of the APC which interacts with cyclin D1 is a Cdc20 protein.

These and other aspects of the invention are set out below.

### BRIEF DESCRIPTION OF THE FIGURES

FIG. 1. Initiation and maintenance of G1 arrest induced by IR. The percentage increase in G1 is shown as the difference in % G1 content between irradiated and control cells.

FIG. 2. Genotoxic stresses induce rapid and specific degradation of cyclin D1 protein.
The estimated half-life of cyclin D1 protein is shown.

FIG. 3. Cyclin D1 degradation after genotoxic stress is independent of GSK3-β.
GSK3-β activity in response to IR is shown.

FIG. 4. A destruction motif in cyclin D1 is required for degradation by genotoxic stress.
(A) Sequence comparison of the cyclin D1 RxxL motif and neighboring amino acids to cyclin D2, D3, E, Ume3p and cyclins A and B.
(B) Half life of wild type and L32A mutant cyclin D1

FIG. 5. Degradation of cyclin D1 is required for initiation of G1 arrest by IR.
(A) Expression of a histone H2B-GFP fusion construct.
(B) Ability of mutants of cyclin D1 to block the initiation of a G1 arrest.
(C) Incorporation of BrdU in MCF-7/E6 cells was used to measure effects on S phase in response to IR.
(D) Examination of the requirement for cyclin D1 degradation in the presence of p53 activity.
(E) S-phase response to IR of primary MEFs lacking cyclin D1.

FIG. 6. Abrogation of cyclin D1 degradation sensitizes to IR.
(A) Survival of cells rendered unable to degrade cyclin D1 in response to IR.
(B) Effect of IR on immortalised MEFs derived from cyclin D1 knockout mice (D1^{-/-}), cyclin E knockin mice (D1^{-/-}-E) and wild type MEFs.

### DETAILED DESCRIPTION OF THE INVENTION

DNA damage inducing agents include ionizing radiation as well as other DNA damaging agents used in chemotherapy, such as cis-platin or anthracyclins such as doxorubicin or its hydrochloride salt, adriamycin. Such agents are widely used in cancer therapy and doses, routes and modes of administration are well understood by the skilled practitioner.

In assays of the invention, the cyclin D1 may be any suitable mammalian cyclin D1, particularly human cyclin D1. Human D1 cyclin has been cloned and sources of the gene can be readily identified by those of skill in the art. See for example, Xiong et al, 1991, Cell 65; 691-699 and Xiong et al, 1992, Genomics 13; 575-84. Murine D1 cyclin has also been cloned. Other mammalian cyclins can be obtained using routine cloning methods analogous to those described in the aforementioned references.

Although wild-type cyclin D1 is preferred, mutants of D1 which still retain the ability to target the cyclin for destruction in response to DNA damage may also be used. Examples of cyclin D1 mutants are well known in the art and a particular mutant is illustrated in the accompanying Examples. For example, the mutant may the cyclin D1-T286A mutant.

It is not necessary to use the entire cyclin D1 proteins for assays of the invention. Fragments of the cyclin may be used provided such fragments retain the RXXL motif described herein and retain the ability to be targeted for destruction in a cell in response to DNA damage. Fragments include N-terminal fragments which retain the CDK4 binding domain as well as the RXXL motif.

Fragments of cyclin D1 may be generated in any suitable way known to those of skill in the art. Suitable ways include, but are not limited to, recombinant expression of a fragment of the DNA encoding the cyclin. Such fragments may be generated by taking DNA encoding the cyclin, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Small fragments of the cyclin (up to about 20 or 30 amino acids) may also be generated using peptide synthesis methods which are well known in the art.

The ability of suitable fragments to be targeted for destruction in response to DNA damage may be tested using routine procedures such as those described in the accompanying examples. Reference herein to cyclin D1 includes the above mentioned mutants and fragments which are functionally able to retain this property, and desirably also retain the ability to bind to activate CDK4 and/or CDK6.

The cyclin D1 may be expressed as a fusion with a marker protein, for example a protein which can be detected via its enzymatic or colourimetric (e.g. fluorescent, luminescent or the like) properties. For example, the cyclin D1 may be fused with green fluorescent protein (GFP) in order to provide a visual marker within a cell. Other marker proteins include chloramphenicol acetyl transferase, luciferase, beta-galactosidase, horseradish peroxidase, and the like.

In a further embodiment, the RXXL motif of the cyclin D1 may be inserted into such a marker protein in order that the marker protein itself is targeted for destruction by a cell in response to DNA damage. The motif may be inserted into the protein in a location so as to retain the activity of the protein, e.g. fluorescence. Those of skill in the art will be able to determine suitable sites, for example between regions of secondary structure or folded domains, as well as the N- and C-termini. One or more of these motifs (e.g. from 2 to 10, such as 2, 3, 4 or 5), which may be the same or different, may be inserted into such proteins, for example at different locations or in tandem.

It will be understood that the identity of the second and third amino acids, “XX” of the motif may be the same or different and may each be any amino acid. Examples of RXXL motifs include RAML, RQKL, RAAL and RTAL. These or other variations may be used. Preferably, the amino acid side chain is non-aromatic and non-cyclic, for example selected from A, G, T, M, S, C, V, L and I.

The motif may be inserted into the marker protein with flanking sequences found in a naturally occurring cyclin D1, for example up to 5, 10 or 20 contiguous residues found N- and/or C-terminal to the motif.

The cyclin D1 or reporter protein will generally be generated within a cell by means of recombinant expression. Vectors for the production of these proteins are illustrated in the accompanying examples, and analogous techniques, which are well known in themselves, may be used by those of skill in the art in providing analogous vectors to produce proteins for assays within the scope of the present invention. Recombinant expression in a cell may be via transient or stable transfection of the cell.

In the abovementioned aspect of the invention which comprises introducing an expressible cDNA into a cell, the cDNA will usually be a member of a cDNA library. Conveniently, the cDNA will be carried by a vector such as a retroviral or adenoviral vector which allows introduction of the cDNA into the cell by infection with a viral particle. In a preferred aspect, the method of the invention will be practised on a multiplicity of members of the cDNA library simultaneously, for example by infecting cells at a multiplicity of infection of 1 virus per cell, and plating said cells into separate wells of microtitre plates, e.g. one or more 96-well plates. The cells will be allowed to grow to provide clonal populations in each well which may then be assayed in accordance with the invention.

cDNA libraries may be provided from a range of species, though most preferably of the species corresponding to the cell type in which the assay of this embodiment of the invention is performed. Mammalian, particularly human, cDNA libraries are preferred. The cDNA libraries may be obtained from a range of tissue sources, including liver, lung, muscle, nerve, brain cells. The cells may be fetal, normal human or tumour cells. An example of the production and use of a retroviral cDNA library may be found in Whitehead et al, 1995, Mol. Cell. Biol., 15; 704-710.

Where the assay of the invention is conducted within a cell, the effect on the degradation of the cyclin D1 or reporter protein (reference henceforth to cyclin D1 in assays of the invention will be understood to include reporter proteins unless specifically indicated to the contrary) may be determined by any suitable means. For example, the amount of the protein may be measured directly, e.g. in the case of a fluorescent reporter by measuring the fluorescence with the cell (or generally a culture of cells), or by immunoassay techniques which determine in a quantitative or qualitative manner the amount of that protein in the cell.

Alternatively, the status of the cell cycle may be observed, for example the cell cycle distribution of cells may be observed, to determine whether the presence of the potential modulator compound has reduced the amount of cells in G1 phase due to the inhibition of cyclin D1 destruction.

It will be appreciated that the above-described assays of the invention will be conducted by reference to suitable controls, which may be either run in parallel with any of the assays, or conducted under a set of reference conditions which are reproduced in the assay, apart from the presence of a potential modulator compound.

In another aspect of the invention, there is provided an assay which relates to the interaction of cyclin D1 protein and the APC or component thereof which interacts with said protein.

It is known in the art that the progression of eukaryotic cells through the cell cycle is controlled by a number of events, including the regulated association of specific cyclins with a CDK (cyclin-dependent-kinase). At the end of mitosis, mitotic cyclin degradation is required. In eukaryotic cells which have been studied, including yeast, Xenopus oocytes and clam oocytes, degradation of cyclin B is mediated by a complex of proteins called the anaphase promoting complex (APC) which functions as a cell cycle-regulated ubiquitin-protein ligase (Zachariae et al, Science, 1996, 274; 1201-1204). The APC is part of the essential cell cycle machinery whose components are evolutionarily conserved (Irniger et al., 1995 Cell, 81, 269-78; King et al., 1995 Cell, 81, 279-88; Tugendreich et al., 1995 Cell, 81, 261-268; Peters et al., 1996 Science, 274, 1199-1201; Zachariae et al., 1996 Science, 274, 1201-4). In yeast CDC16, CDC23, CDC26, CDC27 and APC1 have been identified as genes coding for some of these components (Lamb et al., 1994 EMBO J., 13, 4321-4328; Irniger et al., 1995 ibid; Zachariae et al., 1996, ibid). WO 98/21326 describes the APC complex and methods for analysing components thereof.

Members of the APC include Cdc16 (also referred to as APC6), Cdc23 (also referred to as APC8), Cdc26, Cdc27 (also referred to as APC3), APC1 and APC2.

Such polypeptides may be obtained from a wide variety of sources, including fungi, such as S. cerevisiae or S. pombe, Aspergillus spp and Candida spps, invertebrates such as Drosophila, vertebrates including amphibians such as Xenopus and mammals such as mice and other rodents or primates including humans. The sequences of these proteins are widely available from a number of sources, and vectors encoding these proteins are also available. For example, Sikorski et al, (1993) Mol. Cell Biol., 13, 1212-1221 and (1990) Cell 60, 307-317) disclose Cdc23 from S. cerevisiae and a number of variants thereof, including thermolabile variants. Human cdc23 (APC8) is found on Genbank accession number 3283051 and C. albicans APC8 on plate 396132:A03 Forward of the Candida genome project. Lamb et al (EMBO J., ibid) describe Cdc16, Cdc23 and Cdc27 from S. cerevisiae and their interaction by two-hybrid assay and co-immunoprecipitation. Reference is also made by these authors to sources of Cdc27 from S. pombe, Aspergillus nidulans, Drosophila melanogaster and humans, and to Cdc16 from S. pombe. Cdc27 and Cdc16 activity in Xenopus eggs has been analysed by King et al (Cell, 1995, 81; 279-288). Human Cdc27 and Cdc16 cDNAs are described by Tudendreich et al (Cell, 1995, 81; 261-268). The Cdc16 cDNA was obtained by analysis of an EST database with a known Cdc16 sequence to identify a partial human Cdc16 cDNA sequence, which was then used to construct a full length cDNA. This technique may be used to identify other members of the APC from sources, where such sources are not presently available in the art. Human cdc27 and cdc16 sequences are also identified in U.S. Pat. No. 5,726,025.

APC8 is one of three APC components which comprise multiple copies of a 34 amino acid repeat motif, termed TPR (Hirano et al, 1990 Cell 60, 319-328; Sikorski et al, 1990, ibid), arranged as a block of tandem TPRs in the C-terminus, with one or two additional TPRs in the N-terminus. It has been proposed that TPRs mediate protein-protein interactions (Lamb et al, 1994, ibid) and thus in addition to APC8, cdc16 and cdc27 polypeptides are also of interest as second components in the assay of the invention.

Polypeptides which are fragments, variants and fragments thereof of the APC members may also be used, provided that such polypeptides retain the ability to interact with a cyclin D1 protein, particularly a cyclin D1 protein of the same species as the APC member. Variants and fragments may be made by routine recombinant DNA techniques, as discussed above for the production of cyclin D1.

Thus assays of the present invention include assays in which the interaction between cyclin D1 and the APC is examined within a cell in which the APC has been produced by the cell, as well as assays in which one or more components of the APC are provided as isolated proteins and brought into contact with an isolated cyclin D1 protein, under conditions in which the two proteins, in the absence of a potential modulator, interact.

In the case of the former, the interaction of the cyclin D1 and APC may be determined by means such as detecting one of the two components, for example by immunological means, followed by detecting whether or not the second of these components is associated with the first. For example, as illustrated herein, the interaction is determined by immunoprecipitation of a cell extract using an antibody against the APC subunit Cdc27 followed by immunoblotting the precipitated material to confirm the presence of cyclin D1.

In the case of the latter, the interaction may be determined by providing an isolated component of the APC and the cyclin D1 protein, and directly observing the interaction between the two. Those of skill in the art may select any APC component using routine methodology to determine which, in the absence of a potential modulator compound, provides an interaction which is suitable for detection by the particular assay format selected. For example, the APC component may be selected from any of those mentioned above, such as Cdc16 (also referred to as APC6), Cdc23 (also referred to as APC8), Cdc26, Cdc27 (also referred to as APC3), APC1 and APC2. The component may also be, either alternatively or in addition, an activator of the APC such as a fizzy-related protein, e.g. such as Cdc20 and Hct1.

As indicated above, our experiments have shown that the component of the APC which binds cyclin D1 is a Cdc20 protein. Thus in a preferred embodiment of the assay the APC component is a Cdc20. p55Cdc20 has been sequenced in mammalian cells (Weinstein et al., 1994, Moll Cell Biol, 14(5), 3350-63). Cdc20 is available from humans (GenBank accession number AAH01088), mice (GenBank ref. NP_{—}075712), s. pombe (GenBank ref. T41719), s. cerevisiae (GenBank ref. NP_{—}001246), Atlantic surf clam (GenBank ref. AAC06232, and Tritrichomonas (GenBank ref. AAB5112), and is a homologue of the Xe-fzy, and dm#2-fzy proteins.

The assay may also be performed in the presence of a CDK4. Any suitable CDK4 protein may be used, e.g. a human CDK4 or any other available homologue, e.g. a mammalian, vertebrate or yeast homologue. The CDK4 protein may be an entire wild type CDK4 or a fragment or variant thereof which retains the ability to facilitate the degradation of cyclin D1 via the APC in response to DNA damage.

A variety of assay formats may be used. For example, the interaction between the cyclin D1 polypeptide and the poly-peptide member of the APC may be assayed most directly by tagging one or both of the polypeptides, either in vivo or in vitro, and using the tag as a handle to retrieve the tagged component from a mixture comprising both polypeptides and a putative modulator compound, followed by measuring the amount of other polypeptide which is associated with the retrieved polypeptide.

For example, the interaction between a cyclin D1 polypeptide and an APC polypeptide may be studied by labeling one with a detectable label and bringing it into contact with the other which has been immobilized on a solid support. Suitable detectable labels include ³⁵S-methionine which may be incorporated into recombinantly produced polypeptides. The recombinantly produced polypeptides may also be expressed as a fusion protein containing an epitope which can be labeled with an antibody, such as an antibody immobilized on a solid support.

The protein which is immobilized on a solid support may be immobilized using an antibody against that protein bound to a solid support or via other technologies which are known per se. A preferred in vitro interaction may utilize a fusion protein including glutathione-S-transferase (GST). may be immobilized on glutathione agarose beads. An alternative is to use a histidine tag (e.g. a His6 tag) which may be used to immobilize a polypeptide on Ni++beads. In an in vitro assay format of the type described above the putative modulator compound can be assayed by determining its ability to modulate the amount of labeled first polypeptide which binds to the immobilized GST- or Ni++-second polypeptide. This may be determined by fractionating the glutathione-agarose or Ni++ beads by SDS-polyacrylamide gel electrophoresis.

Alternatively, the beads may be rinsed to remove unbound protein and the amount of protein which has bound can be determined by counting the amount of label present in, for example, a suitable scintillation counter.

Alternatively an antibody attached to a solid support and directed against one of the polypeptides may be used in place of GST to attach the molecule to the solid support. Antibodies against the cyclin D1 and APC polypeptides may be obtained in a variety of ways known as such in the art.

Alternatively, these polypeptides may be in the form of fusion proteins comprising a epitope unrelated to these polypeptides, such as an HA or myc tag. Such antibodies and nucleic acid encoding such epitopes are commercially available.

Other tags may include enzymes, such as horse radish peroxidase, or luciferase, or biotin, avidin or streptavadin.

The interaction between cyclin D1 and an APC polypeptide may be examined by two-hybrid assays (e.g. Fields and Song, 1989, Nature 340; 245-246). In such an assay the DNA binding domain (DBD) and the transcriptional activation domain (TAD) of the yeast GAL4 transcription factor are fused to the first and second molecules respectively whose interaction is to be investigated. Other transcriptional activator domains may be used in place of the GAL4 TAD, for example the viral VP16 activation domain. In general, fusion proteins comprising DNA binding domains and activation domains may be made.

In an alternative mode, one of the cyclin D1 polypeptide and APC polypeptide may be labelled with a fluorescent donor moiety and the other labelled with an acceptor which is capable of reducing the emission from the donor. This allows an assay according to the invention to be conducted by fluorescence resonance energy transfer (FRET). In this mode, the fluorescence signal of the donor will be altered when the polypeptides interact. The presence to a candidate modulator compound which modulates the interaction will increase the amount of unaltered fluorescence signal of the donor.

FRET is a technique known per se in the art and thus the precise donor and acceptor molecules and the means by which they are linked to their respective polypeptides may be accomplished by reference to the literature.

Suitable fluorescent donor moieties are those capable of transferring fluorogenic energy to another fluorogenic molecule or part of a compound and include, but are not limited to, coumarins and related dyes such as fluoresceins, rhodols and rhodamines, resorufins, cyanine dyes, bimanes, acridines, isoindoles, dansyl dyes, aminophthalic hydrazines such as luminol and isoluminol derivatives, aminophthalimides, aminonaphthalimides, aminobenzofurans, aminoquinolines, dicyanohydroquinones, and europium and terbium complexes and related compounds.

Suitable acceptors include, but are not limited to, coumarins and related fluorophores, xanthenes such as fluoresceins, rhodols and rhodamines, resorufins, cyanines, difluoroboradiazaindacenes, and phthalocyanines.

A preferred donor is fluorescein and preferred acceptors include rhodamine and carbocyanine. The isothiocyanate derivatives of these fluorescein and rhodamine, available from Aldrich Chemical Company Ltd, Gillingham, Dorset, UK, may be used to label the polypeptides. For attachment of carbocyanine, see for example Guo et al, J. Biol. Chem., 270; 27562-8, 1995.

Another assay format is dissociation enhanced lanthanide fluorescent immunoassay (DELFIA) (Ogata et al, (1992) J. Immunol. Methods 148(1-2)i 15-22). This is a solid phase based system for measuring the interaction of two macromolecules. Typically one molecule (e.g. the cyclin D1 protein) is immobilised to the surface of a multi-well plate and the other molecule (e.g. the APC component) is added in solution to this. Detection of the bound partner is achieved by using a label consisting of a chelate of a rare earth metal. This label can be directly attached to the interacting molecule or may be introduced to the complex via an antibody to the molecule or to the molecules epitope tag. Alternatively, the molecule may be attached to biotin and a streptavidin-rare earth chelate used as the label. The rare earth used in the label may be europium, samarium, terbium or dysprosium. After washing to remove unbound label, a detergent containing low pH buffer is added to dissociate the rare earth metal from the chelate. The highly fluorescent metal ions are then quantitated by time resolved fluorimetry. A number of labelled reagents are commercially available for this technique, including streptavidin, antibodies against glutathione-S-transferase and against hexahistidine.

Modulator compounds are those which cause the various interactions described herein which form the basis of the present invention to be altered, e.g. agonised or antagonised. The preferred assays of the invention will be designed for antagonists, i.e. inhibitors, of the interactions.

The amount of putative modulator compound which may be added to an assay of the invention will normally be determined by trial and error depending upon the type of compound used. Typically, from about 10 to 200 μM concentrations of putative modulator compound may be used, for example from 50 to 100 μM.

Modulator compounds which may be used may be natural or synthetic chemical compounds used in drug screening programmes. Extracts of plants which contain several characterised or uncharacterised components may also be used. Inhibitor compounds may be provided by way of libraries of commercially available compounds. Such libraries, including libraries made by combinatorial chemical means, are available from companies such as Oxford Asymmetry, Oxford, UK; Arqule Inc, MA, USA; Maybridge Limited, Cornwall, UK, and Tripos UK Limited, Bucks, UK.

A particular class of modulator compounds which may be used are peptides or peptide-mimetics which are based upon the cyclin D1-derived RXXL motif. Thus such peptides, which form a further aspect of the present invention, may comprise at least 4 amino acids, and preferably no more than 50, such as no more than 40, for example no more than 30, or no more than 20 amino acids, e.g. from 4 to 10 amino acids, in which the motif RXXL is present. The two central XX residues may be those exemplified herein above. Such peptides will present the RXXL motif to compete with cyclin D1 in a cell, such that the peptide is capable of down-regulating the response of the cell to DNA damage. Such peptides are preferably based upon the cyclin D1 sequence itself, e.g. are peptide which correspond to a cyclin D1 sequence or have high homology thereto, such as more than 70%, more than 80%, more than 90% or more than 95% amino acid identity. Amino acid identity may be determined by computer based alignment programs, such as BLAST, using default parameters.

A further class of modulator compounds are antibodies which bind to the RxxL motif of cyclin D1, thus interfering with the ability of the APC to initiate destruction of this protein. By “antibodies”, this is meant whole antibodies as well as fragments thereof comprising the variable domains, such as single chain Fvs, Fabs and the like.

A yet further class of modulators are peptides which may be selected, e.g. from peptide display libraries on phage, which bind to the RXXL motif. Such peptides are typically short, e.g. around 5 to 15 amino acids, and have high affinity, being selected from highly diverse libraries.

Modulators such as the peptides and antibodies mentioned above may be used in the course of IR or other therapy in which DNA damage is induced wherein the peptides inhibit cell cycle arrest.

Such a therapy provides for the ability to reduce doses of radiation or chemical agents which cause DNA damage and thus a reduction in potential damage to non-target cells.

Modulators of the invention may be formulated in the form of a salt. Salts of modulators of the invention which may be conveniently used in therapy include physiologically acceptable base salts, eg derived from an appropriate base, such as alkali metal (e.g. sodium), alkaline earth metal (e.g. magnesium) salts, ammonium and NR₄ (wherein R is C₁₋₄ alkyl) salts. Salts also include physiologically acceptable acid addition salts, including the hydrochloride and acetate salts.

Modulators which are peptides or antibodies may be made synthetically or recombinantly, using techniques which are widely available in the art. Synthetic production generally involves step-wise addition of individual amino acid residues to a reaction vessel in which a peptide of a desired sequence is being made.

Modulators of the invention may be in a substantially isolated form. It will be understood that the modulator may be mixed with carriers or diluents which will not interfere with the intended purpose of the modulator and still be regarded as substantially isolated.

The invention also extends to fusion peptides comprising the peptides described above linked at the N- or C-terminus, or both, to further sequence(s). These further sequence(s) may be selected to provide particular additional functions to the resulting fusion peptide. The further sequences do no include sequences which are naturally contiguous to the cyclin D1 peptides.

In general the further sequence(s) will not comprise more than a total of 500 amino acids, optionally split between the N- and C-terminus in any proportion. More desirably the sequences will be much shorter, for example not more than 200, preferably not more than 100, for example not more than 50 or even not more than 20 amino acids in total. The further sequence(s) may be selected by those of skill in the art for a variety of purposes, such as tags (e.g. an HA or myc tag), or membrane translocation sequences capable of directing the fusion peptide through the membrane of a eukaryotic cell.

Modulators may be formulated into pharmaceutical compositions. The compositions comprise the modulator together with a pharmaceutically acceptable carrier or diluent.

Pharmaceutically acceptable carriers or diluents include those used in formulations suitable for oral, topical, or parenteral (e.g. intramuscular or intravenous) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For example, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the modulator to blood components or one or more organs.

The composition may comprise a mixture of more than one, for example two or three, peptides of different sequences having the RXXL motif.

The invention also provides a modulator of the invention and a cytotoxic or cytostatic agent for separate or simultaneous use in the treatment of proliferating cells, for example tumour cells, either in vitro or in vivo.

The invention further provides the use of a modulator of the invention for the manufacture of a medicament for the treatment of proliferating cells wherein said cells are also treated, separately or simultaneously, with a DNA damaging therapy such a chemotherapy or IR.

In a further aspect, the finding that cyclin D1 with a mutant RXXL motif is not destroyed via the APC in response to DNA damage provides a target for gene therapy, e.g. to enhance the response of target cells to DNA damage. Nucleic acids encoding a cyclin D1 in which the RXXL motif has been altered to be non-functional (e.g. by substitution of R or L), particularly when in the form of a recombinant vector, may be used in methods of gene therapy. A construct capable of expressing such nucleic acid may be introduced into cells of a recipient by any suitable means, such that the altered D1 is expressed in the cells.

The construct may be introduced in the form of naked DNA, which is taken up by some cells of animal subjects, including muscle cells of mammalians. In this aspect of the invention the construct will generally be carried by a pharmaceutically acceptable carrier alone. The construct may also formulated in a liposome particle, as described above.

Such methods of gene therapy further include the use of recombinant viral vectors such as adenoviral or retroviral vectors which comprise a construct capable of expressing a polypeptide of the invention. Such viral vectors may be delivered to the body in the form of packaged viral particles.

Constructs of the invention, however formulated and delivered, will be for use in treating tumours in conjunction with therapy. The construct will comprise nucleic acid encoding the altered cyclin D1 linked to a promoter capable of expressing it in the target cells. The constructs may be introduced into cells of a human or non-human mammalian recipient either in situ or ex-vivo and reimplanted into the body. Where delivered in situ, this may be by for example injection into target tissue(s) or in the case of liposomes, inhalation.

Gene therapy methods are widely documented in the art and may be adapted for use in the expression of the altered cyclin D1.

The invention is illustrated by the following examples.

DNA damage causes stabilization of p53, leading to cell cycle arrest through induction of the CDK inhibitor p21^{cip1}. As accumulation of p21^{cip1} by p53 requires transcription, several hours are required to exert this cell cycle inhibitory response. We demonstrate in these examples that in response to ionizing irradiation (IR) cells initiate an immediate and p53-independent G1 arrest, which is caused by proteolysis of cyclin D1. This is mediated through a destruction box in the amino terminus of cyclin D1. The Anaphase Promoting Complex (APC), a genetic link between destruction box-containing proteins and proteolysis in yeast, is potentially involved in IR-induced degradation of cyclin D1, as it is physically associated with the cyclin D1/CDK4 complex. Functionally, destruction of cyclin D1 leads to a release of p21^{cip1} from CDK4 complexes to inhibit CDK2 activity. Interference with cyclin D1 degradation prevents cells from initiating a rapid G1 arrest and renders cells more susceptible to DNA damage. Our results demonstrate that induction of G1-arrest in response to IR is minimally a two step process: a fast induction of G1 arrest mediated by cyclin D1 proteolysis and a slower maintenance of arrest resulting from increased p53 stability.

p53-Independent Initiation of G1 Arrest Induced by IR.

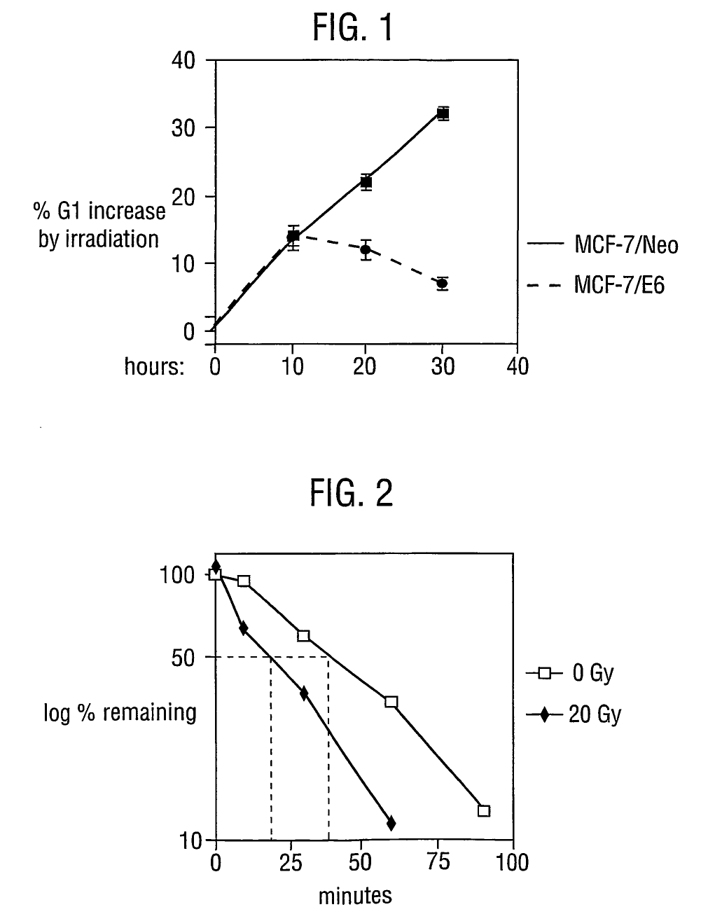

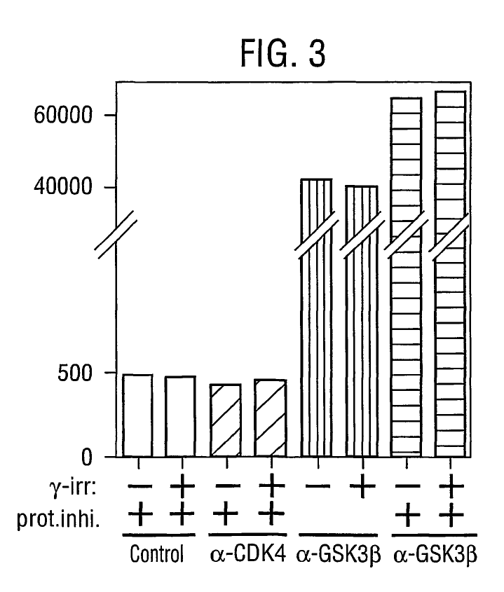

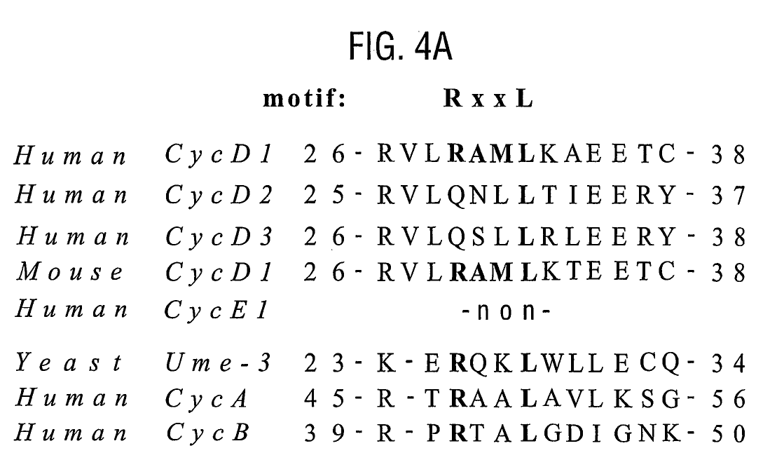

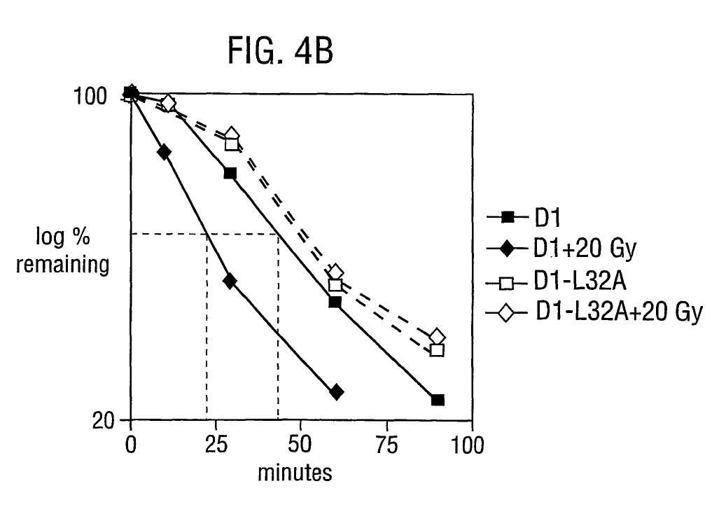

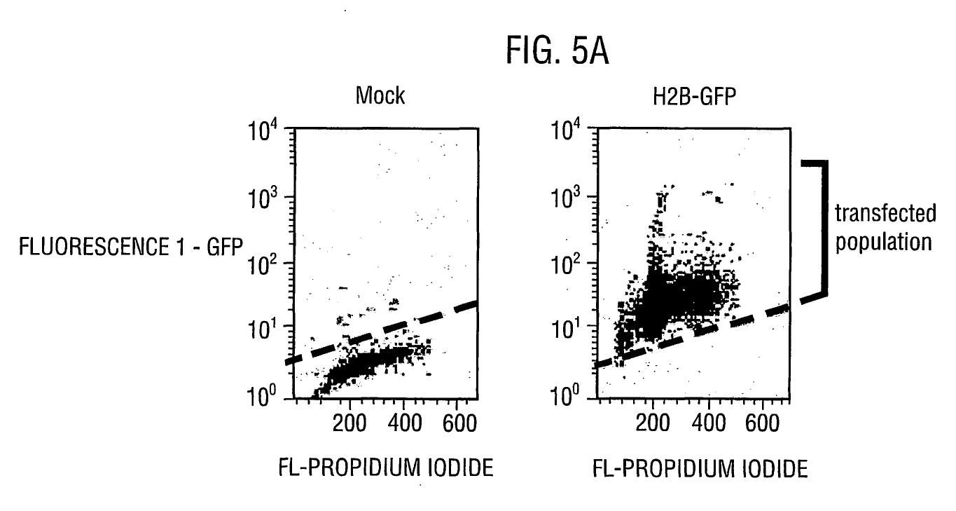

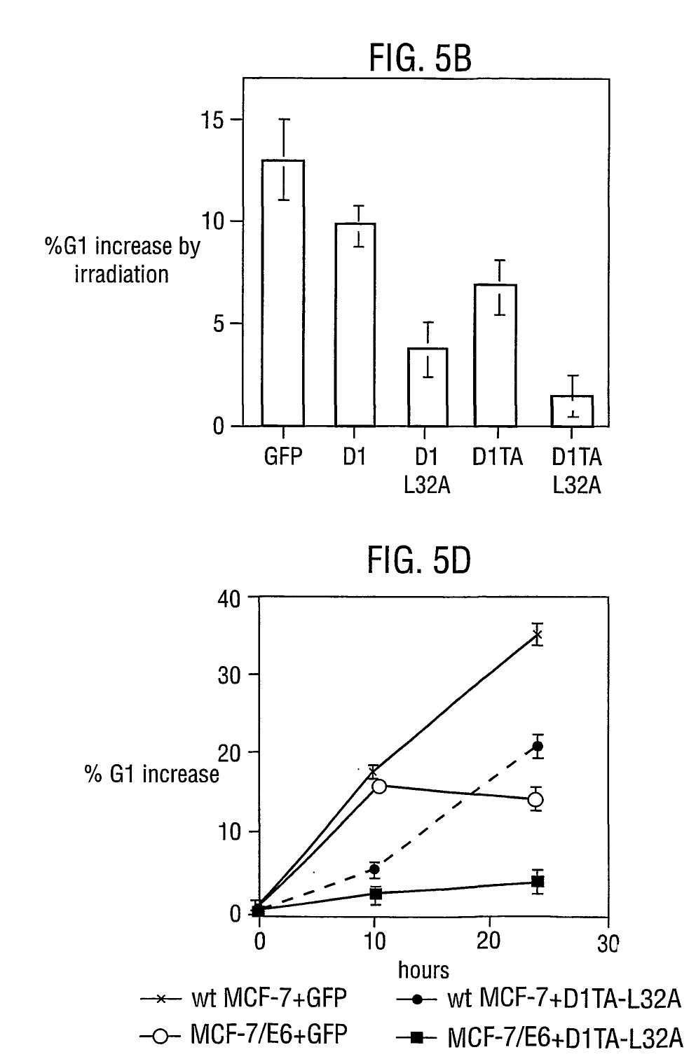

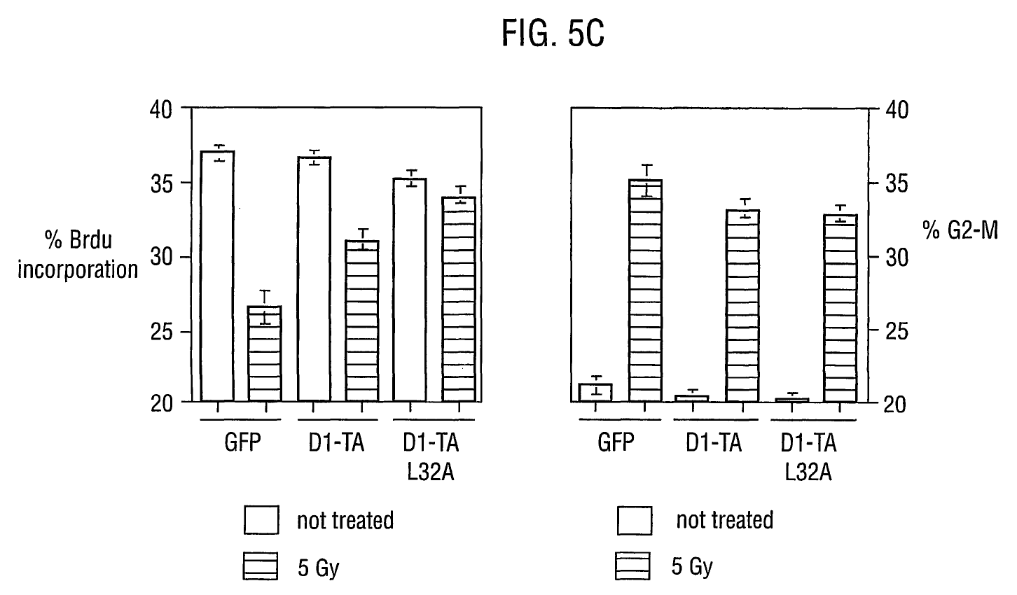

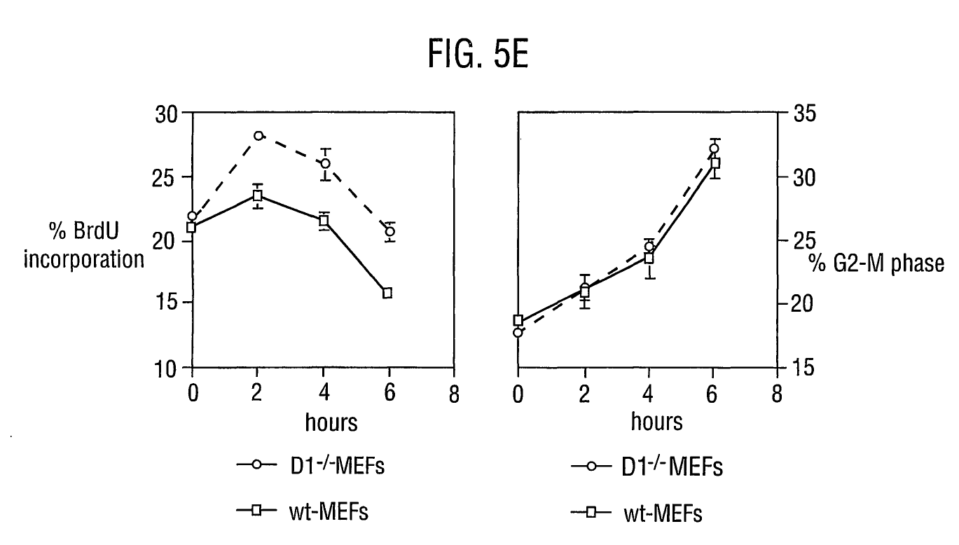

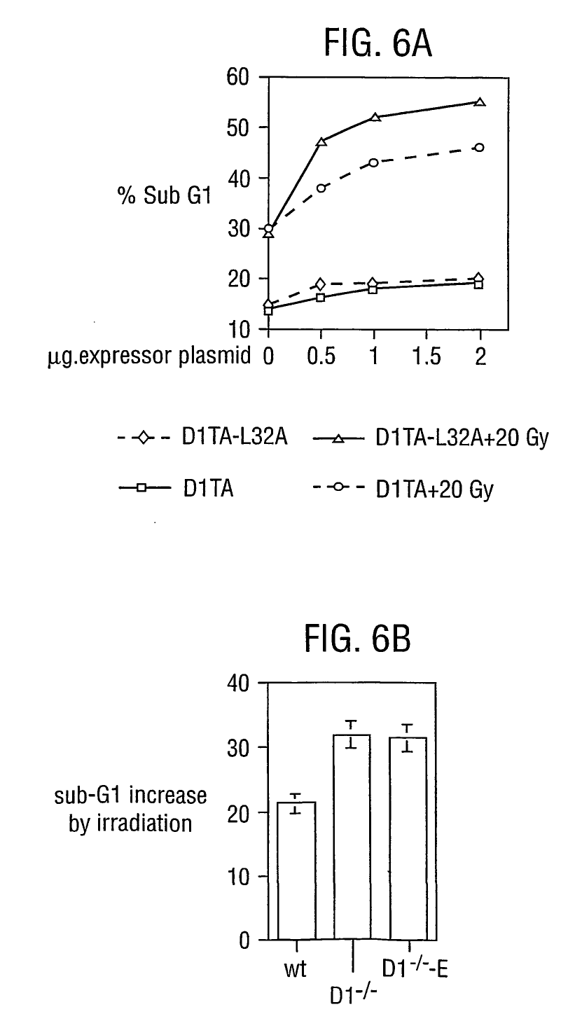

Since the transcriptional response by p53 is a relatively slow process, we asked whether initiation of a G1 arrest following genotoxic stress requires p53. We generated stable MCF-7 clones containing either pCDNA3.1-E6 or pCDNA3.1 (Neo). MCF-7/pCDNA3.1-E6 expresses the HPV16 E6 protein, which mediates degradation of p53 (Scheffner et al., 1990). The MCF-7 clones were irradiated (20Gy) and cellular protein extracts were made two hours later, separated on 10% SDS PAGE, and immunoblotted to detect p53 and cyclin D1 proteins. In the presence of E6, p53 stabilization in response to IR was almost completely prevented in MCF-7 cells. Consistent with this, no induction of p21^{cip1} by IR was seen in the E6-expressing MCF-7 cells. To better visualize the cell cycle effects, we treated irradiated cells with nocodazole, which arrests cells in M phase unless they are arrested in G1 as a result of IR. Close examination of the cellular response of both parental and E6 cells to IR by flow activated cell sorter (FACS) analysis revealed that both exhibited an approximately 15% increase in G1 ten hours after the induction of genotoxic stress (FIG. 1). At twenty and thirty hours after IR, the fraction of parental MCF-7 cells in G1 increased steadily, whereas the E6 cells gradually lost their initial G1 arrest (FIG. 1). This result suggests that cells undergo an initial G1 arrest within 10 hours after exposure to IR and that this initial response does not require p53 activity.

Specific Induction of Cyclin D1 Proteolysis by Genotoxic Stress.

In contrast to p53, we noticed that the cyclin D1 protein level is downregulated both in parental MCF-7 cells and in E6-expressing derivatives within two hours following IR. Downregulation of cyclin D1 was maintained over a period of 24 hours and was not seen both with another G1 cyclin (cyclin E) and the G2/M cyclins A and B1. To study the effects of genotoxic stress on the kinetics of cyclin D1 protein downregulation we exposed U2-OS cells to varying amounts of IR and harvested cells at different time points. Total lysates were analysed by immunoblotting against cyclin D1 and p53 proteins. Exposure to 6 to 20 grays (Gy) resulted in a clear downregulation of cyclin D1 protein levels as early as 10 minutes after IR and a similar effect was seen with 2 Gy after 60 minutes. Compared to the degradation of cyclin D1, the upregulation of p53 was slow following IR. This result shows that in U2-OS cells rapid downregulation of cyclin D1 occurs after IR, which precedes p53 stabilization. Cyclin D1 downregulation occurred with similar kinetics in MCF-7 cells.

We next examined the mechanism underlying the rapid decrease in cyclin D1 protein by genotoxic stress. Northern analysis was carried out on RNA extracted from non-treated and irradiated (20 Gy) MCF-7. At the mRNA level cyclin D1 was slightly elevated at 2 and 4 hours after IR. The effect of IR on cyclin D1 protein expressed from a heterologous CMV promoter was studied. MCF-7 cells were transfected with 2 mg total DNA containing either vector or 0.5 mg CMV promoter based cyclin D1 expression plasmid. Co-transfected GFP construct (0.03 mg) was used to control transfection efficiency. After 48 hours cells were irradiated (20 Gy) and 2 hours later cellular proteins were extracted, separated on 10% SDS PAGE and immunoblotted to detect cyclin D1 and GFP proteins. When expressed from a heterologous CMV promoter, cyclin D1 protein was also downregulated by IR to a similar extent as the endogenous protein. We therefore conclude that transcriptional regulation is not responsible for the cyclin D1 downregulation following IR.

We then asked whether cyclin D1 protein stability was affected in response to IR using a pulse-chase experiment. MCF-7 cells were pulse-labelled with [³⁵S]-methionine and after IR chased with excess cold methionine for the indicated periods of time. Cyclin D1 protein was immunoprecipitated, separated on SDS-PAGE and detected by PhosphorImager. Cyclin D1 was destabilized immediately after IR; its half-life decreased from 40 minutes to less then 20 (FIG. 2). To ask whether the IR-induced degradation of cyclin D1 is mediated by the proteasome, MCF-7 cells were exposed to IR and subsequently the proteasome inhibitor cbz-LLL was added at increasing concentrations for two hours. After two hours, protein lysates were made, separated on 10% SDS PAGE, and Western blotted sequentially with antibodies against cyclin D1, p53 and cyclin E proteins. Even though it was added after exposure to IR, 5 mM cbz-LLL was sufficient to completely block cyclin D1 downregulation without any effect on cyclin E protein levels. Cyclin D1 was also rapidly degraded in response to other genotoxic agents such as cis-platin. Collectively, these results indicate strongly that accelerated proteolysis induced by genotoxic stress is the main mechanism responsible for the rapid downregulation of cyclin D1 protein.

We then asked if cyclin D1 degradation after genotoxic stress is common to many cell types and is uncoupled from cell cycle progression. HeLa, HPV16-containing cervical carcinoma; CAPAN, SEK1-mutated pancreas carcinoma; SW1417, SEK1 mutated colon carcinoma; AT-1BR, primary fibroblasts from AT patient; MEF, p19^{ARF -/-} mouse embryo fibroblasts; T47D and ZR75-1, breast carcinoma with low and high level of cyclin D1, respectively; U2-OS, osteosarcoma cells were subjected to treatments with 20 Gy IR and 10 mM proteasome inhibitor as above. SaOS-2 osteosarcoma were either transfected with 0.1 and 0.5 mg cyclin D1 construct. Co-transfected GFP construct was used to control transfection efficiency. After 48 hours cells were IR (20 Gy) and two hours later cellular proteins were extracted, separated on 10% SDS PAGE and immunoblotted to detect cyclin D1 and GFP proteins. Genotoxic stress-induced cyclin D1 degradation was seen in a variety of cell lines, with SaOS-2 osteosarcoma cells being the only exception to date. Since transfected cyclin D1 protein did not degrade following IR either, it is clear that the inability of SaOS-2 cells to degrade cyclin D1 does not involve alterations in the cyclin D1 itself. Cyclin D1 degradation also occurred both in HeLa cells that do not arrest in G1 following IR due to the presence of the HPV EG and E7 proteins and in U2-OS cells which were growth arrested artificially by the induction of p19^{ARF} with muristerone-A. We therefore conclude that mechanistically, cyclin D1 degradation after genotoxic stress is uncoupled from cell cycle progression. Moreover, cyclin D1 degradation could occur in cell lines that lack functional p16^{INK4A}, p19^{ARF}, pRb and p53 proteins and the ATM and SEK1 kinases and does not depend on these proteins.

Remarkably, exposure to IR of cells which express apart from cyclin D1 also the closely related cyclins D2 or D3 (Mouse Embryo Fibroblasts (MEFs) and HeLa), revealed that IR-induced degradation was unique to cyclin D1.

Cyclin D1 Degradation by Genotoxic Stress is Independent of the GSK-3β Pathway.

Activation of the PI3K-PKB/Akt-GSK-3β pathway leads to cyclin D1 degradation through phosphorylation of threonine 286 of cyclin D1 by GSK3-β (Diehl et al., 1998). We therefore asked whether this pathway is also activated by IR and is involved in stress-induced degradation of cyclin D1. To investigate the co-immunoprecipitation of GSK3-β with CDK4-cyclin D1 complex, MCF-7 cells were subjected to treatment with proteasome inhibitor cbz-LLL and IR. 5% of the cell lysates or the immunoprecipitated protein complexes were separated on 10% SDS-PAGE and immunoblotted against cyclin-D1, CDK4, GSK3- and control JNK1 proteins. GSK3-β was found to be specifically associated with the CDK4/cyclin D1 complex in the co-immunoprecipitation experiments. However, the amount of GSK3-β bound to CDK4/cyclin D1 was not significantly increased in response to IR. We used proteasome inhibitors to protect cyclin D1 from degradation thereby making a direct comparison between the different treatments possible. FIG. 3 shows that neither the total cellular activity of GSK3-β kinase nor the GSK3-β activity associated with CDK4 was elevated by IR. To further investigate whether the GSK3-β pathway is involved in the degradation of cyclin D1 by IR we treated irradiated cells with Li⁺ ions, as Li⁺ has been shown to inhibit all GSK3 activity in cells (Stambolic et al., 1996). MCF-7 cells were treated with increasing concentrations of LiCl or control KCl and subsequently IR (20 Gy). Lysates were prepared after 2 hours, separated on 10% SDS-PAGE and immunoblotted sequentially with anti-cyclin D1 and anti-p53 antibodies. If this pathway is involved, Li⁺ ions should inhibit cyclin D1 degradation. Results showed that Li⁺ ions had no detectable effect on cyclin D1 degradation by IR although, as expected, an increase in cyclin D1 levels was seen in non-irradiated cells due to inhibition of basal GSK3-β activity (Diehl et al., 1998). Finally, a mutant of cyclin D1 in which the GSK3-β phosphorylation site was mutated (T286A), which is completely refractory to GSK3-β induced degradation (Diehl et al., 1998), was fully responsive to IR-induced degradation. Collectively, these results strongly suggest that cyclin D1 degradation induced by genotoxic stress is independent of the PI3K-PKB/Akt-GSK3β pathway.

Cyclin D1 Degradation by Genotoxic Stress Requires a RxxL Destruction Motif.

To map the motif in cyclin D1 that mediates its degradation by genotoxic stress we analyzed several mutants of D1 by expression in MCF-7 cells. In all these experiments a co-transfected GFP construct was used to confirm equal transfection efficiencies between irradiated- and control cells. When cyclin D1 was mutated at a site within the cyclin box that is essential for activation of CDK4/6 (mutant K112E), D1 degradation by IR remained. The same result was obtained when the pRb family binding site in cyclin D1 was mutated (LxCxE mutant). We therefore conclude that D1 induced degradation by genotoxic stress is independent of both CDK4/6 kinase and pRb binding.

In the yeast Saccharomyces cerevisiae, degradation of the cyclin C homologue Ume3p can be induced by various stress signals such as heat, oxidative stress and ethanol shock (Cooper et al., 1997; Cooper et al., 1999). Three regions in Ume3p are required for stress-induced degradation, including a destruction box at the amino terminus (RxxL motif), the amino terminal region of the cyclin box and a PEST domain. Close inspection of the cyclin D1 protein sequence revealed that cyclin D1, but not cyclin D2 and D3, harbors a destruction box-like motif in its N-terminus (FIG. 4A). Since cyclin D2 is not degraded by the genotoxic stress response we mutated cyclin D1 to the corresponding amino acid in cyclin D2. We found that point mutations within the amino terminal region of the cyclin box (amino acids 87 to 99) had no effect on the degradation by IR. However, two independent point mutations within the putative destruction box of cyclin D1 (either R29Q or L32A) completely abolished degradation by IR. Combining each of these mutations in the destruction box with a mutation in the GSK3-β phosphorylation site (R29Q; T286A and L32A; T286A mutants) gave rise to a higher level of protein expression in non-irradiated cells that was fully resistant to the IR effect, in sharp contrast to the T286A single mutant. These data suggest that the RxxL destruction box in cyclin D1 is the major motif that renders cyclin D1 susceptible to degradation by IR. To further investigate this, we performed a pulse-chase experiment with the cyclin D1 L32A destruction box mutant to determine its half-life. MCF-7 cells were transfected by electroporation with wild type or L32A mutant cyclin D1 expression vector, pulse-labelled with [³⁵S]-methionine and chased for varying periods of time with excess cold methionine. FIG. 4B shows a graphic representation of the results of this experiment, which indicates that the wild type and L32A mutant cyclin D1 have a comparable half-life in non-irradiated cells of about 50 minutes. This is comparable to that of endogenous cyclin D1 protein (FIG. 2). Significantly, the L32A mutant cyclin D1 protein was not destabilized in response to IR, whereas the wild type protein was (FIG. 4B). Taken together, these results define the destruction motif at amino acids 29 to 32 as necessary for cyclin D1 degradation by genotoxic stress, but not for its normal metabolic turnover.

To ask whether this motif is sufficient to mediate degradation in response to IR we transplanted it to the non-responsive cyclin D2 protein. MCF cells were transfected with either wild type or mutant D2 expression plasmids. The effect of irradiation on cyclin D2-RAMLK mutant, in which the amino acids at positions 29-33 were changed to resemble the cyclin D1 RXXL motif, was studied. After 48 hours cells were IR (20 Gy) and two hours later cellular proteins were extracted, separated on 10% SDS PAGE and immunoblotted to detect cyclin D2, cyclin D2-RAMLK and GFP proteins. Co-transfected GFP construct was used to control transfection efficiency. Remarkably, changing four amino acids in cyclin D2, thereby creating the cyclin D1 RxxL motif, converted it to a genotoxic stress degradable cyclin. This result demonstrates that the RxxL motif of cyclin D1 is necessary and, when placed in the context of a D-type cyclin, also sufficient to mediate degradation in response to genotoxic stress.

The role of the motif was further investigated by expression of a fusion protein in which GFP was expressed in a fusion with cyclin D1. It was found that this fusion protein was also targeted for degradation. Such a fusion protein provided an efficient and simple read out of the degradation of the protein which contains the D1-derived destruction box.

Specific Interaction of Cyclin-D1/CDK4 Complex with the APC.

Destruction boxes are conserved motifs (consensus: RxxL) found in mitotic cyclins subject to proteolytic cleavage by a multi-component ubiquitin protein ligase, named the Anaphase-Promoting Complex (APC). Since cyclin D1 harbors a destruction box-like motif, we searched for an association of endogenous cyclin D1/CDK4 complexes with Cdc27, a conserved component of the APC (King et al., 1995).

In a first experiment, whole cell extracts of MCF-7 cells were immunoprecipitated with either an antiserum raised against the APC subunit Cdc27 or a control anti-p38 antibody. The presence of CDK4, cyclin D1 and Cdc27 proteins was detected by immunoblotting. In non-transfected MCF-7 cells we clearly and specifically detected both endogenous CDK4 and cyclin D1 proteins in Cdc27 immunoprecipitates.

In a second experiment, MCF-7 cells were irradiated (20 Gy), and one hour later, cells were harvested and protein lysates were prepared. Subsequently, extracts were immunoprecipitated with either anti-cyclin D1 or control antibodies and subjected to immunoblotting against cdc27, cyclin D1 and CDK4 proteins. Cdc27 was found to be present in cyclin D1 inmmoprecipitates.

In a third experiment, MCF-7 cells were treated with 20 Gy IR and 10 mM proteasome inhibitor cbz-LLL and harvested one hour later. Immunoprecipitation and immunoblotting were carried out as above. Cdc27 was found to be present in anti-CDK4, but not anti-CDK2, immunoprecipitates. Significantly, the interaction between CDK4 and Cdc27 was not affected by IR, whereas the amount of Cdc27 bound to cyclin D1 decreased, most likely due to degradation of cyclin D1 by IR. These results indicate that the APC is constitutively associated with the cyclin D1/CDK4 complex and are consistent with a model in which the APC is responsible for cyclin D1 proteolysis in response to IR.

Cyclin D1 Degradation is Required to Initiate G1 Arrest Induced by IR.

We wished to address the role of cyclin D1 degradation in the initiation of G1 arrest by genotoxic stress. Our strategy was to abolish IR-induced cyclin D1 degradation using transient over-expression of the IR-non-degradable mutant (D1-L32A). In transient transfections, the cyclin D1-T286A (TA) mutant was reproducibly expressed at higher levels than wild type cyclin D1. Therefore, to compete more efficiently with the relatively high level of endogenous cyclin D1 in MCF-7 cells, we performed most of the next experiments using the double mutant T286A; L32A as a genotoxic stress-resistant protein and the D1-T286A mutant as a degradable control. In these experiments we transiently introduced expression vectors into cells using electroporation (see experimental procedures). The advantage of this method is that we reproducibly obtained more than 90% transient transfection efficiencies with very homogeneous expression of the introduced vectors. This is demonstrated by expression of a histone H2B-GFP fusion construct (FIG. 5A). Here MCF-7 cells were transfected by electroporation with 2 mg DNA containing either vector or 0.5 mg histone H2B-GFP expression construct. After 17 hours cells were washed, to clear dead cells, and after additional 48 hours collected and analyzed by FACS). This allowed us to perform experiments without selection of the transfected population.

To assess the ability of mutants of cyclin D1 to block the initiation of a G1 arrest, we focused first on MCF-7/E6 cells since they initiate a G1 response to IR, which is indistinguishable from parental MCF-7 cells, but have no effects originating from p53. We electroporated MCF-7/E6 cells with wild type or mutant cyclin D1 expression vectors and after 48 hours, cells were irradiated, treated with nocodazole and 10 hours later the cell cycle distribution was analyzed by FACS. FIG. 5B shows that the initiation of a G1 arrest of control GFP-transfected MCF-7/E6 cells to IR was similar to non-transfected population (induction of 15% G1 increase, FIG. 5B). Cells transiently transfected with the IR-non-degradable mutants D1-L32A and D1-T286A; L32A had only an increase of 4% and 2% in G1 phase cells in response to IR, respectively. The double mutant D1-T286A; L32A was most efficient in blocking the IR induced G1 arrest, most likely because of its efficient accumulation in cells. The residual 2% G1 increase in the D1TA-L32A transfected population may be the result of the fact that we did not transfect 100% of the population (FIG. 5A). Over-expression of the IR-degradable D1 and D1TA mutant proteins gave a partial effect on G1 increase (FIG. 5B), probably because not all of the overexpressed protein was degraded.

In a second experiment, to measure effects on S phase in response to IR, MCF-7/E6 cells were transfected as in B and 48 hrs later were IR (5 Gy). After additional 9 hours 7.5 mg/ml BrdU was added and cells were harvested 1 hour later, fixed, stained with anti-BrdU and FITC conjugated goat-anti-mouse antibodies and analyzed by FACS. We observed approximately a 10% reduction of cells in S-phase ten hours after IR (FIG. 5C). Over-expression of D1TA-L32A gave complete resistance to the IR-induced S phase decrease, but did not affect the initial G2/M arrest (FIG. 7C). These results suggest strongly that in the absence of a functional p53 DNA damage checkpoint, the initial G1 arrest in response to IR is the result of rapid cyclin D1 degradation.

We then examined the requirement for cyclin D1 degradation in the presence of p53 activity. Parental MCF-7 and MCF-7/E6 cells were transfected with 1 mg of the plasmid cyclin D1TA-L32A, or mock-transfected with GFP as described above. Similar to untreated parental MCF-7 cells, mock-transfected cells induced about 15% and 35% G1 arrest in response to 10 Gy IR after 10 and 24 hours, respectively (FIGS. 1 and 5D). MCF-7 cells, transiently transfected with cyclin D1TA-L32A were unable to efficiently initiate G1 arrest at 10 hours (4-5% G1 increase). However, between 10 and 24 hours, these cells induced a G1 arrest with comparable kinetics as the mock-transfected cells, indicating that the slow response was to a large extent unaffected. The opposite effect was seen in the E6-expressing cells: the initiation of G1 arrest was normal but the slower response (after 10 hours) was affected (FIGS. 1 and 7D). Consistent with these data, transient over-expression of D1TA-L32A in MCF-7/E6 abrogated both the initial and the slower G1 arrest functions (FIG. 7D). These results indicate that MCF-7 cells respond to IR by activating two distinct and independent pathways. They initiate G1 arrest through a process that depends on the ability of cells to degrade cyclin D1 and later on they maintain and further strengthen it by stabilizing p53.

In a further experiment primary wild type and cyclin D1^{-/-} MEFs were irradiated (10 Gy) and harvested after 2 hours. Whole cell extracts were prepared and analyzed by SDS-PAGE immunoblotting procedure using antibodies against cyclin D1. In agreement with a role for cyclin D1 in the initiation of G1 arrest following IR, results showed that the S-phase response to IR of primary MEFs lacking cyclin D1 is defective when compared to wild type MEFs. Wild type and D1^{-/-} cells were irradiated (10 Gy) and harvested at between 0, 2, 4 and 6 hours. 1 hour before harvesting, 7.5 mg/ml BrdU was added and cells were analyzed by FACS (FIG. 5E). Cyclin D1 knockout MEFs consistently had higher fraction of S phase cells in the first hours after IR than control wild type MEFs, whereas no effect was observed on the induction of G2/M block immediately after stress (FIG. 5E).

Cyclin D1 Degradation by Genotoxic Stress Induces a Rapid Redistribution of p21^{cip1} from CDK4 to CDK2.

One mechanistic explanation as to how cyclin D1 degradation can cause a fast G1 cell cycle arrest is by release of CKIs from CDK4 to inhibit CDK2 complexes. To investigate this, parental MCF-7 and MCF-7/E6 cells were irradiated and harvested one hour later. Whole cell extracts were immuno-precipitated with anti-CDK4, anti-CDK2 or control anti-p38 antibodies. 10% of the total extracts and the immunoprecipitates were separated on 12% SDS-PAGE. To distinguish between mechanisms involving proteolytic cleavage and others we examined IR effects also in the presence of 10 mM of the proteasome inhibitory agent cbz-LLL. Analysis of extracts of both cell types by sequential immunoblotting, with anti-cyclin D1, anti-p21^{cip1}, anti-p27^{kip1}, anti-CDK4, anti-CDK2 and control anti-p38 antibodies, revealed that the level of p21^{cip1} in MCF-7/E6 was only somewhat reduced compared to parental cells. This observation is in line with previous observations that p53 has a limited effect on basal p21^{cip1} levels in cells (Macleod et al., 1995; Parker et al., 1995).

In co-immunoprecipitation experiments using both cell types, we observed that in non-irradiated cells, more cyclin D1 was associated to CDK4 than to CDK2. Upon exposure to IR, cyclin D1 level was reduced both in CDK4- and CDK2 protein complexes, a process that could be blocked by proteasome inhibitor. This indicates that genotoxic stress-induced cyclin D1 degradation is the main mechanism to initiate its disappearance from CDK2 and CDK4 complexes. Most importantly, we could clearly detect that p21^{cip1} dissociated from CDK4 and started to accumulate in CDK2 complexes, even at this early time point, a process that was also dependent on proteolysis. In contrast, p27^{kip1} was associated with CDK4 in non-irradiated cells and it did not redistribute to CDK2 complexes upon IR. We therefore detect an early p53-independent and proteasome-dependent, redistribution of p21^{cip1}, but not of p27^{kip1} from CDK4 complexes to CDK2.

We next determined the CDK2 activity precipitated from MCF-7/E6 cells treated with IR. Cells were treated as above, except that cells were harvested after 2 hours. Using histone H1 as a substrate we found that IR markedly reduced CDK2 activity after two hours, which could be blocked by treatment with proteasome inhibitor. Identical results were obtained with parental MCF-7 cells. Therefore, protein degradation seems to be necessary for fast CDK2 kinase inhibition after genotoxic stress.

To examine the role of cyclin D1 degradation in the process of p21^{cip1} redistribution and CDK2 inhibition we analyzed CDK4 complexes from cells transfected, by electroporation, with the IR-non-degradable D1-TA-L32A mutant. MCF-7/E6 cells were mock-tranfected or tranfected with with 1 mg of H2B-GFP, D1-TA, or D1-TA-L32A as described in the previous example. After 48 hours cells were irradiated (20 Gy) and 1 hour later whole cell extracts were prepared and subjected to co-immunoprecipitation with anti-CDK4 and control anti-p38 antibodies. 5% of each extract and the immunoprecipitated complexes were separated on 12% SDS-PAGE and immunoblotted against p21^{cip1}, cyclin D1 and CDK4. Consistent with the results described above, already one hour after IR we detected efficient removal of cyclin D1 from CDK4 complexes. Over-expression of the D1-TA in cells increased the amount of cyclin D1 bound to CDK4 in non-irradiated cells, which was reduced in irradiated cells. However, due to the higher pre-IR levels, more cyclin D1 remained bound to CDK4 after IR as compared to either mock or H2B-GFP-transfected cells. In sharp contrast, the IR-non-degradable D1 mutant (TA-L32A) remained associated with CDK4 after IR and almost no p21^{cip1} was released from CDK4 complexes by DNA damage. This result demonstrates that p21^{cip1} dissociation from CDK4 complexes in response to IR requires cyclin D1 degradation.

We then examined the CDK2 activity in response to IR of cells transiently over-expressing either D1TA or D1TA-L32A proteins. MCF-7/E6 cells were electroporated as above, irradiated (20 Gy) and harvested 2 hours later. CDK2 protein was immunoprecipitated and its kinase activity was examined using Histone 1 as a substrate (H1). CDK2 protein level was determined by immunoblotting (IB) of the same membrane with an antibody against CDK2. Two hours after IR inhibition of CDK2 activity in mock-transfected cells was comparable to non-transfected cells. In contrast, in response to IR CDK2 activity remained unchanged in cells expressing the IR-non degradable D1TA-L32A.

Collectively, these results demonstrate that initiation of G1 arrest by IR is a result of the ability of cells to degrade cyclin D1. Degradation of cyclin D1 is required to inhibit CDK2 activity by redistribution of p21^{cip1} from CDK4 complexes to CDK2. However, we can not rule out that other processes that are influenced by cyclin D1 degradation, are involved as well.

Cyclin D1 Degradation is Required for Cellular Resistance to Genotoxic Stress

Next, we determined the survival of cells that were rendered unable to degrade D1 in response to IR. MCF-7 cells were transiently transfected with the IR-non-degradable cyclin D1TA-L32A construct at increasing concentrations. Cells were washed 17 hours after transfection and exposed to IR (20 Gy) after an additional 24 hours. Five days after irradiation, floating and adherent cells were harvested and analyzed for their sub-G1 content by FACS. FIG. 6A shows that expression of cyclin D1TA-L32A significantly increased cell death in response to IR in a concentration dependent fashion (up to 22% more cell death). This occurred with very limited toxicity of cyclin D1TA-L32A on untreated cells (5% more cell death). In a second experiment immortalized MEFs of either wild type (wt), cyclin D1-knockout (D1^{-/-}) or cyclin E knockin into the cyclin D1 locus (D1^{-/-}-E) origins were exposed to IR (10 Gy) and harvested 6 days later for FACS analysis (FIG. 6B). Consistent with a critical role for cyclin D1 in DNA damage response, immortalized MEFs derived from cyclin D1 knockout mice (D1^{-/-}) were more sensitive to IR as compared to wild type immortalized MEFs (10% more cell death, FIG. 6B).

Significantly, immortalized MEFs derived from D1^{-/-} mice which express cyclin E under the control of the cyclin D1 promoter (cyclin E knockin mice, (Geng et al., 1999)), were also more sensitive to IR (D1^{-/-}-E, FIG. 6B). Collectively, these data indicate that cyclin D1 degradation is an essential component of the cellular response to genotoxic stress, in the absence of which the cell's ability to deal with DNA damage is compromised.

Initiation and Maintenance of G1 Arrest by Genotoxic Stress.

Genotoxic stresses, such as IR, induce a fast and strong G1 arrest that is sustained over a prolonged period of time. We report here that this type of G1 arrest builds up in two different and mechanistically distinct phases: initiation and maintenance. The initial process is fast (accomplished in a period of less than ten hours), strong (more than 15% increase in G1 in an asynchronous population) and is mediated by cyclin D1 degradation. p53 activity is dispensable for G1 arrest in this initial period. At a later stage, p53 activity is required to maintain and further strengthen the initial p53-independent G1 arrest. These distinct mechanisms collaborate to allow the cell to achieve a fast and sustained G1 arrest in response to IR.

Judging from the speed at which cyclin D1 is degraded by genotoxic stress (FIG. 2), it appears that all factors required to mediate cyclin D1 degradation are pre-existing in the cell. Such pre-existing machinery is well-suited to carry out a quick response to genotoxic stress. In contrast, the G1 arrest established by activation of the p53 pathway is indirect and involves p53 protein accumulation by de novo protein synthesis, its translocation to the nucleus, transcriptional activation of p53 target genes such as p21^{cip1}, translation of p53-induced transcripts, and accumulation of the induced proteins to sufficiently high levels that they affect the cell cycle. This p53 response depends on several time-consuming processes and is therefore inherently slow. Therefore, the p53 response appears more suited to maintain and further strengthen an already established G1 arrest, rather than to initiate it. This notion is supported by the present data, which show that p53 hardly contributes to G1 arrest in the first 10 hours after exposure to IR.

Our results suggest strongly that the initial phase of G1 arrest following IR relies primarily on downregulation of cyclin D1 protein levels. Several lines of experimental evidence support the notion that induced proteolysis is the main mechanism used by irradiated cells to reduce cyclin D1 protein levels. First, treatment of cells with IR caused a significant decrease in cyclin D1 protein stability (FIG. 2). Second, treatment of cells with specific inhibitors of the proteasome completely blocked cyclin D1 downregulation by IR. Third, downregulation of cyclin D1 is mediated through a destruction box, a motif that is involved in proteolytic destruction of mitotic cyclins (FIG. 4A). Fourth, mutation of the cyclin D1 destruction box rendered the protein non-degradable by IR, whereas transplantation of the cyclin D1 destruction box to the IR-non-degradable cyclin D2 protein, rendered cyclin D2 unstable in response to IR (FIG. 4B). Finally, in cells treated with both IR and proteasome inhibitor, cyclin D1 accumulated to higher levels than non-treated cells. Together, these results indicate that exposure to IR triggers a rapid proteolysis of cyclin D1 and virtually exclude the possibility that IR also controls cyclin D1 other levels, such protein translation.

Genotoxic Stress Versus Mitogen Deprivation.

Cyclin D1 plays a role in relaying mitogenic signals to the cell cycle machinery. When cells are deprived of mitogens, cyclin D1 is phosphorylated at threonine 286 by GSK3-b and targeted for nuclear export and proteolysis (Diehl et al., 1998; Diehl et al., 1997). Stimulation of cell cycle entry by mitogens activates the PI3K-PKB/Akt pathway, which inhibits GSK3-b activity, leading to accumulation of cyclin D1 in the nucleus. Similar to mitogen deprivation, genotoxic stresses induce cyclin D1 degradation. However, this is accomplished through a different and independent pathway. First, genotoxic stress-induced cyclin D1 degradation occurs both in cycling cells and in arrested cells with similar efficiencies (FIG. 3). Second, GSK3-b is neither activated by IR nor involved in genotoxic stress-mediated cyclin D1 degradation (FIG. 3). Third, both signals converge on different protein motifs in cyclin D1. Whereas the mitogenic signals are mediated by phosphorylation of cyclin D1 at threonine 286, genotoxic stress requires an intact RxxL destruction box motif (amino acids 29-32) within cyclin D1.

It is noteworthy that the three D-type cyclins differ in their sensitivity to genotoxic stress-induced degradation. Proteolytic degradation by genotoxic stress was specific to cyclin D1 and was not observed with its homologues cyclin D2 or D3 (FIG. 4). Consistent with this, the RxxL motif is not conserved in these cyclins. This may suggest that under physiological conditions the D type cyclin family can modulate cellular response to the various external signals. Whereas cyclin D1 will mediate efficient responses to both mitogen deprivation and genotoxic stress, cyclin D2 will respond only to the former.

Our data by no means rule out the possibility that the specific degradation machinery responsible for cyclin D1 degradation by genotoxic stress also targets other proteins that may function in other genotoxic stress responses such as apoptosis, repair or G2-M arrest. It will therefore be important to identify the requirements and essential consensus amino acid motif that mediates the specific interaction with the relevant proteolytic machinery.

Rapid p21^{cip1} Redistribution and Inhibition of CDK2 Activity.

Genetic experiments with mice in which the cyclin E gene was placed under the control of the cyclin D1 promoter have suggested that cyclin E is a downstream target of cyclin D1 (Geng et al., 1999; Roberts, 1999; Sicinski et al., 1995). Our data are consistent with such a model in that degradation of cyclin D1 in G1 by IR immediately affects cyclin E-associated kinase activity. IR significantly inhibits cyclin E-CDK2 activity within two hours. Remarkably, we find that the initial inhibition of CDK2 activity depends almost exclusively on the cellular proteolytic activity and more specifically on the ability to degrade cyclin D1. Blocking either proteasome-mediated proteolysis or specifically cyclin D1 proteolysis was sufficient to abrogate completely CDK2 inhibition by IR. Moreover, we demonstrate that cyclin D1 degradation initiates.

A specific release of p21^{cip1} from CDK4 complexes immediately after IR, a process that culminates in a rapid increase of p21^{cip1} associated with cyclin E/CDK2 and inhibition of its kinase activity. However, in the absence of p53 this effect was not sufficient to maintain cells in G1 20 to 30 hours after IR (FIG. 1), even though low levels of cyclin D1 protein were maintained at that time. The escape of cells with non-functional p53 from the initial G1 arrest probably stems from the fact that the reservoir of p21^{cip1} held by cyclin D1/CDK4 complex is quickly exhausted in response to IR. Consequently, newly synthesized CDK2/cyclin E complexes will be active and able to drive cells into S phase. In cells harboring wild type p53, activation of newly synthesized cyclin E/CDK2 will be prevented through induction of p21^{cip1} expression by p53.

In contrast to p21^{cip1}, which was rapidly released from CDK4 upon exposure to IR, p27^{kip1} remained bound to CDK4. We do not know what the molecular basis is for this specific redistribution of inhibitors by genotoxic stress. This situation is clearly different from the redistribution of CKI family proteins from CDK4/6 induced by TGF-b. In this case induction of one of the INK4 family members efficiently competes for binding of both p21^{cip1} and p27^{kip1} to cyclin D-CDK4/6 complexes.

The RxxL Destruction Motif and the APC.

Induction of cyclin D1 degradation by genotoxic stress requires a RxxL motif at the amino terminus of cyclin D1. RxxL motifs, also known as destruction boxes, have been studied most extensively in mitotic cyclins. The sea urchin cyclin B must be degraded for cells to exit mitosis, which is dependent on a nine amino acid motif including the RxxL box (Glotzer et al., 1991). Likewise, the Anaphase-Promoting Complex (APC), a multimeric ubiquitin ligase complex of 1.5 MDa, is essential for mitotic cyclin degradation through their destruction box (Irniger et al., 1995; King et al., 1996). The specificity and timing of proteolysis by the APC is determined by phosphorylation and association with activating proteins of the fizzy protein family such as Cdc20 and Hct1 (Lukas et al., 1999; Schwab et al., 1997; Sigrist and Lehner, 1997; Visintin et al., 1997). Which components of the APC direct the specificity of binding to RxxL motifs is unknown.

Interestingly, during cell cycle progression, APC carries out its major role in exit from M phase, but remains active in G1 and G0 when mitotic kinases are no longer active (Amon et al., 1994; Brandeis and Hunt, 1996). This suggests possible roles for APC in G1 and G0 phases of the cell cycle as well. Our identification of the RxxL destruction motif as a necessary element for cyclin D1 degradation points to the involvement of APC in this process. Strongly supporting this view is the fact that the cyclin D1/CDK4 complex specifically associates with the APC in cycling cells. Whereas the interaction of APC with CDK4 remains intact in cells exposed to IR, the interaction with cyclin D1 decreases rapidly. Therefore, it seems that CDK4 serves as a bridging factor between cyclin D1 and the APC. This suggests a model in which the APC marks cyclin D1 for proteolysis and is subsequently free to bind another cyclin D1 molecule via CDK4.

The APC in Response to DNA Damage

To identify which proteins within the APC complex are required for cyclin D1 destruction following genotoxic stress we looked at the human p55Cdc20 protein, an activator of the APC member of the fizzy protein family. We looked at Cdc20 protein in MCF-7 cells two hours after exposure to IR and found that its mobility was slightly shifted, an indication for modification and possibly regulation.

Furthermore, as discussed above, cyclin D1 degradation by IR occurs in many cell lines and cell types, except for human Saos-2 osteosarcoma cells. Since exogenously introduced cyclin D1 was also not subject to degradation by IR in Saos-2 cells, a likely explanation is that this cell type lacks an upstream component in the pathway. We therefore monitored the Cdc20 protein level in Saos-2 cells, and found that Cdc20 is hardly or not at all expressed in Saos-2 cells when compared to MCF-7 or a number of other cell-types. This effect was specific to Cdc20 as Cdc27, a core component of the APC, is equally expressed in both cell types. The level at which Cdc20 expression is hampered is yet to be determined.

To test the functional requirement of Cdc20 for cyclin D1 destruction by IR we cloned Cdc20 that was amplified by PCR from a human cDNA library into a mammalian expression vector and re-introduced it into Saos-2 cells. We then selected stable clones and examined Cdc20 and cyclin D1 protein levels in response to IR. Expression of Cdc20 was clearly detected in two clones, 8 with low expression and 9 with expression similar to the levels seen in MCF-7 cells. Importantly, re-expression of Cdc20 restored significant cyclin D1 degradation in response to IR.

To investigate the possible interaction between Cdc20 and cyclin D1 we employed in vitro GST pull-down assays. We labelled either the full-length Cdc20 protein or a truncated form, containing only the seven WD40 motifs, with ³⁵S Methionine using the reticulocyte lysate system. These proteins were then incubated with purified GST-cyclin D1 protein produced in bacteria and immobilized on beads. A clear and specific interaction of both Cdc20 proteins with cyclin D1 was detected. Furthermore, this specific interaction was retained when only the first 85 amino acids of cyclin D1, containing its RxxL destruction box fused to GST, were used. Taken together, these results indicate that the human p55Cdc20, an activator of the APC, is a crucial component responsible for conducting the response from DNA damage to destruction of cyclin D1 via the APC and direct interaction.

Induction of Cyclin D1 Degradation by Genotoxic Stress and Cancer.

The p16^{INK4A}-cyclin D1-pRb pathway is disrupted in most, if not all, human tumors. In a substantial number of tumors cyclin D1 is over-expressed by mechanisms involving gene amplification, chromosomal translocations, transcriptional activation or defects in proteolysis (Hanahan and Weinberg, 2000). Interestingly, we find that cyclin D1-induced degradation by genotoxic stress is intact in the vast majority of cell lines examined. Moreover, it occurs both in the presence and absence of the main genes involved in tumorigenesis (p53, pRb, p16^{INK4A} and p19^{ARF}). The finding that the genotoxic stress-induced cyclin D1 degradation pathway is intact in most tumor cells may be related to the fact that disruption of this pathway will not elevate cyclin D1 protein levels in non-stressed cells and therefore does not confer a selective advantage to tumor cells.

We show that activation of cyclin D1 proteolytic cleavage by genotoxic stresses occurs in a broad range of cell types and is conserved from man to mouse. Our findings also have potential relevance for treatment of cancer. We demonstrate that abrogation of genotoxic stress-induced cyclin D1 degradation sensitizes cells to genotoxic stress with no significant effect on survival of non-irradiated cells (FIG. 6). This result suggests that specific inhibition of genotoxic stress induced-cyclin D1 degradation could make chemotherapy and radiotherapy more effective and selective as tumor cells often express much higher levels of cyclin D1 than the surrounding normal tissue.

Experimental Procedures

Materials, Antibodies and Plasmids Construction

Cis-platin was purchased from Teva. Histone H1 and the proteasome inhibitor cbz-LLL were purchased from Sigma. IR was done with a 2×415 Ci ¹³⁷Cs source.

For Western blot and co-immunoprecipitation analyses the antibodies used in this study were anti-human p53 (Do-1), anti-mouse p53 (FL-393), anti-cyclin D1 (H-295 and M-20), anti-human cyclin D2 (C-17), anti-mouse cyclin D2 (M−20), anti-cyclin D3 (C-16), anti-cyclin E (M-20), anti-CDK4 (H-22), anti-CDK2 (M-2), anti-p21^{cip1} (C-19), anti-JNK1 (FL) and anti-p38 (C-20) from Santa Cruz. Other antibodies used were anti-GSK3-b mAb (Transduction lab.), anti-Kip1/p27 mAb (Transduction lab.), anti-Cdc27 mAb (Transduction lab.), rabbit anti-p19^{ARF} (ABCAM) and rabbit-anti-GFP (made in house).

The plasmids pRC-CMV-cyclin D1 and the mutants K112E and LxCxE were described (Zwijsen et al., 1997). pRC-CMV cyclin D2 clone was described (Dowdy et al., 1993). Cyclin D1 mutants, T286A, E92V, R98H, R29Q, L32A and cyclin D2-RAMLK were generated by site directed mutagenesis using polymerase chain reactor (PCR) and were cloned in the pCDNA3.1 vector (Clontech). The double mutants R29Q-T286A and L32A-T286A were generated by conventional cloning using an internal unique BssHII site in cyclin D1 cDNA. All constructs and mutants were verified by DNA sequence analysis. The plasmid used for green florescent protein (GFP) expression was pEGFP (Clontech). H2B-GFP has also been described (Kanda et al., 1998). For pIND-p19^{ARF} construct the mouse p19^{ARF} cDNA tagged with HA (Quelle et al., 1995) was cloned into the pIND vector (Invitrogen).

Cell Transfection

Cell transfection was carried out in two ways. In FIGS. 1 to 5, MCF-7 cells were either transiently or stably transfected with DOTAP (Boehringer Mannheim). Transient transfection experiments presented in FIGS. 5 and 6 were done using electroporation. Here, 3×10⁵ MCF-7 cells were resuspended in 100 ml of electroporation buffer containing 2 mM Hepes pH: 7.2, 15 mM K₂HPO₄/KH₂PO₄, 250 mM manitol and 1 mM MgCl₂ at a final pH of 7.2. Either one or two mg of DNA was added and the cells and DNA were transferred to a 0.1 cm electroporation cuvette (BioRad) and electroporated with Gene Pulser II apparatus and Gene Pulser II RF module (BioRad) at 140 volts, 15 times 1.5 msec burst duration and 1.5 sec intervals. Five minutes after electroporation, cells were seeded in a 10 cm dish. Cells were washed 16 hours after transfection and the experiment was preformed either 24 or 48 hours later.

To generate the MCF-7/Neo and MCF-7/E6 stable clones, cells were transfected with either pCDNA3.1 or the HPV16 E6 construct and selection with 750 mg/ml of G418 was carried out for 2 weeks. Selected clones were tested by immunoblot analysis. The pIND-p19^{ARF}stable inducible U2-OS clone was generated using the Ecdysone system (Invitrogen) and will be described in more detail elsewhere. Gene induction was done with 1 mM Muristerone-A (Invitrogen) for 20 hours.

Immortalization of Primary MEFs

Primary MEFs were immortalized using infection with a LZRS virus caring the Bmi-1 cDNA which downregulates expression of the INK4a locus (Jacobs et al., 1999).

Cell Cycle Profile Analysis

For FACS analysis cells were trypsinized and resuspended in 600 ml solution containing 0.6% NP-40, 50 mg/ml RNaseA and 50 mg/ml propidium iodide in PBS. In each assay ten thousand cells were collected by FACScan (Becton Dickinson) and analyzed with the CellQuest program (Becton Dickinson). For Bromodeoxyuridine (BrdU) labelling, cells were incubated 1 hour prior to the harvest with 7.5 mg/ml BrdU. After harvest, cells were fixed in ethanol and stained sequentially with mouse anti-BrdU antibodies (DAKO) and FITC-conjugated goat-anti-mouse-antibodies (MONOSAN) according to a standard protocol (Boehringer Mannheim).

For determination of sub-G1 population MCF-7 cells were transfected by electroporation, as described above, and irradiated (20 Gy) after 24 hours. Five days later, floating and adherent cells were harvested and analyzed by FACScan. Determination of sub-G1 population in wt and D1^{-/-} MEFs was done similarly only that cells were irradiated (10 Gy) and analyzed six days later.

Pulse-Chase Experiments

MCF-7 cells were starved in Dulbecco's modified Eagle's medium (DMEM) without methionine and cysteine containing 5% dialyzed serum for 1 hour and then were metabolically labelled with L-[³⁵S] methionine and L-[³⁵S] cysteine for 2 hours. Subsequently cells were treated with IR (20 Gy) and chased in DMEM containing 5% serum for the indicated time periods. Cells were lysed in lysis buffer containing 50 mM Hepes pH: 7.4, 0.1% NP-40, 250 mM NaCl, 10 mM b-glycerophosphate, 0.5 mM sodium vanadate, 0.5 mM DTT and protease inhibitor cocktail (Complete, Boehringer Mannheim) for 20 min at 4 C and centrifuged for 15 min at 4 C. Protein samples were pre-cleared with protein A-sepharose beads for 20 min at 4 C, immunoprecipitated with the anti-cyclin D1 (H-295) antibody for 1 hr at 4 C and washed three times with RIPA buffer (150 mM NaCl, 1% NP-40, 0.5% DOC, 0.1% SDS and 50 mM Tris: pH 8.0). Fifty ml SDS-sample buffer was added, samples were boiled for 5 min and 20 ml were resolved on 10% SDS-PAGE. The gel was dried, treated with fixation solution for 30 min and protein amounts were quantified with PhosphorImager (BAS-2000, Fuji).

Co-Immunoprecipitation Experiments

Cells (two 80% confluent 10 cm dishes per treatment) were collected and lysed in 500 ml lysis buffer for 30 minutes on ice and then 500 ml of lysis buffer without NaCl was added. Extracts were centrifuged at 14,000 rpm for 15 minutes at 4 C and immunoprecipitated for 1 hour at 4 C in total volume of 800 ml with 200 ml of 10% slurry protein A-sepharose beads (Pharmacia Tech.) pre-conjugated to 2 mg of the specific antibody. The beads were washed five times and the bound proteins were eluted by boiling in SDS-sample buffer and resolved by 12% SDS-PAGE.

For Co-immunoprecipitation of cyclin D1 and CDK4 with APC, MCF-7 cells (80% confluent 10 cm dish per treatment) were extracted and immunoprecipitated as described previously (Agami et al., 1999). Immunoprecipitations were carried out using rabbit antiserum against Cdc27 (Kramer et al., 1998), anti-CDK4 (H-22), anti-cyclin D1 (M-20) and the controls anti-Abl (K-12), Anti-CDK2(M-2) and anti-p38 antibodies. Immunoblotting was done using the mouse monoclonal anti-Cdc27 (Transduction lab.) and rabbit polyclonals anti-cyclin D1 (H-295) and anti-CDK4 (H-22).

In-Vitro Immunoprecipitation-Kinase Assays.

To determine CDK2 activity, specific complexes from either MCF-7/Neo or MCF-7/E6 cells were immunoprecipitated from extracts using anti-CDK2 antibody (M-2). The beads were washed two additional times with kinase buffer (20 mM Tris HCl pH:7.4, 4 mM MgCl₂ and 0.5 mM DTT) and kinase reaction was carried out in 50 ml volume kinase buffer containing 10 mg histone-H1 as a specific substrate, 10 mCi [γ-³²P]-ATP (5000 mCi/mmol, Amersham) and 30 mM ATP at 37 C for 30 minutes. GSK3-β activity, was determined exactly as described in (van Weeren et al., 1998) using peptide PG-S1 as a substrate.

Legends to Figures

FIG. 1. Initiation and Maintenance of G1 Arrest Induced by IR.

Stable MCF-7 clones containing either pCDNA3.1 (Neo) or pCDNA3.1-E6 were irradiated (10 Gy) and after 30 min 1 mg/ml nocodazole was added. At the indicated time points after IR cells were harvested and analyzed by flow activated cell sorter (FACS). Untreated cells (nt) were harvested at the 10 hour time point. Each experiment was carried out in duplicate. The percentage increase in G1 is the difference in % G1 content between irradiated and control cells.

FIG. 2. Genotoxic Stresses Induce Rapid and Specific Degradation of Cyclin D1 Protein.

Endogenous cyclin D1 was immunoprecipitated from MCF-7 cells that were metabolically labelled, IR (20 Gy) and chased for the indicated time points. Cyclin D1 was visualized with PhosphoImager and quantified. The estimated half-life of cyclin D1 protein is shown.

FIG. 3. Cyclin D1 Degradation after Genotoxic Stress is Independent of GSK3-β.

GSK3-β activity in response to IR. MCF-7 cells were IR (20 Gy) and treated with 10 mM proteasome inhibitor cbz-LLL, as indicated. Lysates were prepared and subjected to co-immunoprecipitation with either anti-CDK4, anti-GSK3-β or control anti-JNK1. GSK3-β kinase activity was determined as described (van Weeren et al., 1998).

FIG. 4. A Destruction Motif in Cyclin D1 is Required for Degradation by Genotoxic Stress.

(A) Sequence comparison of the cyclin D1 RxxL motif and neighboring amino acids to cyclin D2, D3, E, Ume3p and cyclins A and B. (B) Half life of wild type and L32A mutant cyclin D1. MCF-7 cells were transfected by electroporation (see FIG. 5A) with 4 mg of wild type cyclin D1 or 6 mg of the L32A mutant and divided into five 3 cm dishes. After 60 hrs cells were pulse-labelled. Typically, 3-4 folds cyclin D1 expression over endogenous protein was obtained.

FIG. 5. Degradation of Cyclin D1 is Required for Initiation of G1 Arrest by IR.

(A) Expression of a histone H2B-GFP fusion construct. Transfected population is indicated and reproducibly was higher than 90%. (B) Ability of mutants of cyclin D1 to block the initiation of a G1 arrest. MCF-7/E6 cells were electroporated with 1 mg of the indicated constructs. After 48 cells were irradiated (10 Gy), treated with nocodazole and 10 hours later the cell cycle distribution was analyzed by FACS. A summary of the observed percentage G1 increase on irradiation, from three independent experiments, is shown. (C) Incorporation of BrdU in MCF-7/E6 cells was used to measure effects on S phase in response to IR. Bars represent two independent experiments in duplicates. (D) Examination of the requirement for cyclin D1 degradation in the presence of p53 activity. Parental MCF-7 and MCF-7/E6 cells were transfected with 1 mg of the indicated plasmids as described in 5A and the experiment was done as described in 5B. A summary of two independent experiments in duplicates is shown. (E) S-phase response to IR of primary MEFs lacking cyclin D1. Wild type and D1^{-/-} cells were irradiated (10 Gy) and harvested at the indicated time points. 1 hour before harvesting, 7.5 mg/ml BrdU was added and cells were analyzed by FACS. Bars represent two independent experiments in duplicates.

FIG. 6. Abrogation of Cyclin D1 Degradation Sensitizes to IR.

(A) Survival of cells rendered unable to degrade cyclin D1 in response to IR. Parental MCF-7 cells were electroporated with increasing amounts of cyclin D1TA or D1TA-L32A mutant constructs as described above. Apoptic cell death was scored as the sub-G1 fraction in a FACS analysis. (B) Effect of IR on immortalised MEFs derived from cyclin D1 knockout mice (D1^{-/-}), cyclin E knockin mice (D1^{-/-}-E) and wild type MEFs. Cell death was scored as above.

### REFERENCES

Agami, R., Blandino, G., Oren, M., and Shaul, Y. (1999). Interaction of c-Abl and p73alpha and their collaboration to induce apoptosis. Nature 399, 809-13.
Agami, R. and Bernards, R. (2000). Distinct initiation and maintenance mechanisms cooperate to induce G1 cell cycle arrest in response to DNA damage. Cell, 102(1), 55-66.
Adams, P. D., Sellers, W. R., Sharma, S. K., Wu, A. D., Nalin, C. M. and Kaelin, W. G. (1996). Identification of a cyclin-cdk2 recognition motif present in substrates and p21-like cyclin-dependent kinase inhibitors. Mol Cell Biol, 16(12), 6623-33.
Amon, A., Irniger, S., and Nasmyth, K. (1994). Closing the cell cycle circle in yeast: G2 cyclin proteolysis initiated at mitosis persists until the activation of G1 cyclins in the next cycle. Cell 77, 1037-50.
Bouchard, C., Thieke, K., Maier, A., Saffrich, R., Hanley-Hyde, J., Ansorge, W., Reed, S., Sicinski, P., Bartek, J., and Eilers, M. (1999). Direct induction of cyclin D2 by Myc contributes to cell cycle progression and sequestration of p27. Embo J 18, 5321-33.
Brandeis, M., and Hunt, T. (1996). The proteolysis of mitotic cyclins in mammalian cells persists from the end of mitosis until the onset of S phase. Embo J 15, 5280-9.
Bunz, F., Dutriaux, A., Lengauer, C., Waldman, T., Zhou, S., Brown, J. P., Sedivy, J. M., Kinzler, K. W., and Vogelstein, B. (1998). Requirement for p53 and p21 to sustain G2 arrest after DNA damage. Science 282, 1497-501.
Chan, T. A., Hermeking, H., Lengauer, C., Kinzler, K. W., and Vogelstein, B. (1999). 14-3-3Sigma is required to prevent mitotic catastrophe after DNA damage. Nature 401, 616-20.
Cooper, K. F., Mallory, M. J., Smith, J. B., and Strich, R. (1997). Stress and developmental regulation of the yeast C-type cyclin Ume3p (Srb11p/Ssn8p). Embo J 16, 4665-75.
Cooper, K. F., Mallory, M. J., and Strich, R. (1999). Oxidative stress-induced destruction of the yeast C-type cyclin Ume3p requires phosphatidylinositol-specific phospholipase C and the 26S proteasome. Mol Cell Biol 19, 3338-48.
Diehl, J. A., Cheng, M., Roussel, M. F., and Sherr, C. J. (1998). Glycogen synthase kinase-3beta regulates cyclin D1 proteolysis and subcellular localization. Genes Dev 12, 3499-511.
Diehl, J. A., Zindy, F., and Sherr, C. J. (1997). Inhibition of cyclin D1 phosphorylation on threonine-286 prevents its rapid degradation via the ubiquitin-proteasome pathway. Genes Dev 11, 957-72.
Dowdy, S. F., Hinds, P. W., Louie, K., Reed, S. I., Arnold, A., and Weinberg, R. A. (1993). Physical interaction of the retinoblastoma protein with human D cyclins. Cell 73, 499-511.
Draetta, G. F. (1994). Mammalian G1 cyclins. Curr Opin Cell Biol 6, 842-6.
el-Deiry, W. S., Tokino, T., Velculescu, V. E., Levy, D. B., Parsons, R., Trent, J. M., Lin, D., Mercer, W. E., Kinzler, K. W., and Vogelstein, B. (1993). WAF1, a potential mediator of p53 tumor suppression. Cell 75, 817-25.
Geng, Y., Whoriskey, W., Park, M. Y., Bronson, R. T., Medema, R. H., Li, T., Weinberg, R. A., and Sicinski, P. (1999). Rescue of cyclin D1 deficiency by knockin cyclin E. Cell 97, 767-77.
Glotzer, M., Murray, A. W., and Kirschner, M. W. (1991). Cyclin is degraded by the ubiquitin pathway. Nature 349, 132-8.
Hanahan, D., and Weinberg, R. A. (2000). The hallmarks of cancer. Cell 100, 57-70.
Irniger, S., Piatti, S., Michaelis, C., and Nasmyth, K. (1995). Genes involved in sister chromatid separation are needed for B-type cyclin proteolysis in budding yeast. Cell 81, 269-78.
Jacobs, J. J., Kieboom, K., Marino, S., DePinho, R. A., and van Lohuizen, M. (1999). The oncogene and Polycomb-group gene bmi-1 regulates cell proliferation and senescence through the ink4a locus. Nature 397, 164-8.
Kanda, T., Sullivan, K. F., and Wahl, G. M. (1998). Histone-GFP fusion protein enables sensitive analysis of chromosome dynamics in living mammalian cells. Curr Biol 8, 377-85.
Kastan, M. B., Onyekwere, O., Sidransky, D., Vogelstein, B., and Craig, R. W. (1991). Participation of p53 protein in the cellular response to DNA damage. Cancer Res 51, 6304-11.
Kastan, M. B., Zhan, Q., el-Deiry, W. S., Carrier, F., Jacks, T., Walsh, W. V., Plunkett, B. S., Vogelstein, B., and Formace, A. J., Jr. (1992). A mammalian cell cycle checkpoint pathway utilizing p53 and GADD45 is defective in ataxia-telangiectasia. Cell 71, 587-97.
King, R. W., Deshaies, R. J., Peters, J. M., and Kirschner, M. W. (1996). How proteolysis drives the cell cycle. Science 274, 1652-9.
King, R. W., Peters, J. M., Tugendreich, S., Rolfe, M., Hieter, P., and Kirschner, M. W. (1995). A 20S complex containing CDC27 and CDC16 catalyzes the mitosis-specific conjugation of ubiquitin to cyclin B. Cell 81, 279-88.
Kramer, E. R., Gieffers, C., Holzl, G., Hengstschlager, M., and Peters, J. M. (1998). Activation of the human anaphase-promoting complex by proteins of the CDC20/Fizzy family. Curr Biol 8, 1207-10.
Kuerbitz, S. J., Plunkett, B. S., Walsh, W. V., and Kastan, M. B. (1992). Wild-type p53 is a cell cycle checkpoint determinant following irradiation. Proc Natl Acad Sci USA 89, 7491-5.
Lipinski, M. M., and Jacks, T. (1999). The retinoblastoma gene family in differentiation and development. Oncogene 18, 7873-82.
Livingstone, L. R., White, A., Sprouse, J., Livanos, E., Jacks, T., and Tlsty, T. D. (1992). Altered cell cycle arrest and gene amplification potential accompany loss of wild-type p53. Cell 70, 923-35.
Lukas, C., Sorensen, C. S., Kramer, E., Santoni-Rugiu, E., Lindeneg, C., Peters, J. M., Bartek, J., and Lukas, J. (1999). Accumulation of cyclin B1 requires E2F and cyclin-A-dependent rearrangement of the anaphase-promoting complex. Nature 401, 815-8.
Macleod, K. F., Sherry, N., Hannon, G., Beach, D., Tokino, T., Kinzler, K., Vogelstein, B., and Jacks, T. (1995). p53-dependent and independent expression of p21 during cell growth, differentiation, and DNA damage. Genes Dev 9, 935-44.
Morgan, D. O. (1995). Principles of CDK regulation. Nature 374, 131-4.
Morin, P. J. (1999). beta-catenin signaling and cancer. Bioessays 21, 1021-1030.
O'Neill, T., Dwyer, A. J., Ziv, Y., Chan, D. W., Lees-Miller, S. P., Abraham, R. H., Lai, J. H., Hill, D., Shiloh, Y., Cantley, L. C. and Rathbun, G. A. (2000). Utilization of oriented peptide libraries to identify substrate motifs selected by ATM. J Biol Chem, 275(30), 22719-27. Parker, S. B., Eichele, G., Zhang, P., Rawls, A., Sands, A. T., Bradley, A., Olson, E. N., Harper, J. W., and Elledge, S. J. (1995). p53-independent expression of p21^{cip1} in muscle and other terminally differentiating cells. Science 267, 1024-7.
Perez-Roger, I., Kim, S. H., Griffiths, B., Sewing, A., and Land, H. (1999). Cyclins D1 and D2 mediate myc-induced proliferation via sequestration of p27(Kip1) and p21(Cip1). Embo J 18, 5310-20.
Quelle, D. E., Zindy, F., Ashmun, R. A., and Sherr, C. J. (1995). Alternative reading frames of the INK4a tumor suppressor gene encode two unrelated proteins capable of inducing cell cycle arrest. Cell 83, 993-1000.
Roberts, J. M. (1999). Evolving ideas about cyclins. Cell 98, 129-32.
Scheffner, M., Werness, B. A., Huibregtse, J. M., Levine, A. J., and Howley, P. M. (1990). The E6 oncoprotein encoded by human papillomavirus types 16 and 18 promotes the degradation of p53. Cell 63, 1129-36.
Schwab, M., Lutum, A. S., and Seufert, W. (1997). Yeast Hct1 is a regulator of Clb2 cyclin proteolysis. Cell 90, 683-93.
Sherr, C. J. (1994). G1 phase progression: cycling on cue [see comments]. Cell 79, 551-5.
Sherr, C. J. (1995). Mammalian G1 cyclins and cell cycle progression. Proc Assoc Am Physicians 107, 181-6.
Sherr, C. J., and Roberts, J. M. (1999). CDK inhibitors: positive and negative regulators of G1-phase progression. Genes Dev 13, 1501-12.
Sherr, C. J., and Roberts, J. M. (1995). Inhibitors of mammalian G1 cyclin-dependent kinases. Genes Dev 9, 1149-63.
Sicinski, P., Donaher, J. L., Parker, S. B., Li, T., Fazeli, A., Gardner, H., Haslam, S. Z., Bronson, R. T., Elledge, S. J., and Weinberg, R. A. (1995). Cyclin D1 provides a link between development and oncogenesis in the retina and breast. Cell 82, 621-30.
Sigrist, S. J., and Lehner, C. F. (1997). Drosophila fizzy-related down-regulates mitotic cyclins and is required for cell proliferation arrest and entry into endocycles. Cell 90, 671-81.
Sionov, R. V., and Haupt, Y. (1999). The cellular response to p53: the decision between life and death. Oncogene 18, 6145-57.
Stambolic, V., Ruel, L., and Woodgett, J. R. (1996). Lithium inhibits glycogen synthase kinase-3 activity and mimics wingless signalling in intact cells [published erratum appears in Curr Biol 1997 Mar. 1; 7(3):196]. Curr Biol 6, 1664-8.
Tetsu, O., and McCormick, F. (1999). Beta-catenin regulates expression of cyclin D1 in colon carcinoma cells. Nature 398, 422-6.
van Weeren, P. C., de Bruyn, K. M., de Vries-Smits, A. M., van Lint, J., and Burgering, B. M. (1998). Essential role for protein kinase B (PKB) in insulin-induced glycogen synthase kinase 3 inactivation. Characterization of dominant-negative mutant of PKB. J Biol Chem 273, 13150-6.
Visintin, R., Prinz, S., and Amon, A. (1997). CDC20 and CDH1: a family of substrate-specific activators of APC-dependent proteolysis. Science 278, 460-3.
Waldman, T., Kinzler, K. W., and Vogelstein, B. (1995). p21 is necessary for the p53-mediated G1 arrest in human cancer cells. Cancer Res 55, 5187-90.
Weinert, T. (1998). DNA damage and checkpoint pathways: molecular anatomy and interactions with repair. Cell 94, 555-8.
Weinstein, J., Jacobsen, F W., Hsu-Chen, J., Wu, T., Baum, LG., (1994). A novel mammalian protein p55CDC, present in dividing cells is associated with protein Kinase activity and has homology to the Saccharomyces cerevisiae cell division cylce proteins Cdc20 and Cdc4. Mol Cell Biol 14(5), 3350-63.
Yin, Y., Tainsky, M. A., Bischoff, F. Z., Strong, L. C., and Wahl, G. M. (1992). Wild-type p53 restores cell cycle control and inhibits gene amplification in cells with mutant p53 alleles. Cell 70, 937-48.
Zwijsen, R. M., Wientjens, E., Klompmaker, R., van der Sman, J., Bernards, R., and Michalides, R. J. (1997). CDK-independent activation of estrogen receptor by cyclin D1. Cell 88, 405-15.
Zwijsen, R. M., Buckle, R. S., Hijmans, E. M., Loomans, C. J. and Bernards, R. (1998). Ligand-independent recruitment of steroid receptor coactivators to estrogen receptor by cyclin D1. Genes Dev, 12(22), 3488-98.

1. An assay for a modulator of cell cycle control, which assay comprises:
(a) providing a cell in culture together with a potential modulator compound, said cell expressing a cyclin D1 which undergoes degradation in response to DNA damage;
(b) exposing said cell to a DNA damaging agent; and
(c) determining the extent to which the presence of the potential modulator compound inhibits the degradation of said cyclin D1.

2. An assay for a modulator of cell cycle control, which assay comprises:
(a) providing a cell in culture together with a potential modulator compound, said cell expressing a reporter protein having an RXXL destruction box and which protein undergoes degradation in response to DNA damage;
(b) exposing said cell to a DNA damaging agent; and
(c) determining the extent to which the presence of the potential modulator compound inhibits the degradation of said reporter protein.

3. An assay which comprises:
(a) providing a cell in culture, said cell expressing a cyclin D1 which undergoes degradation in response to DNA damage;
(b) introducing into said cell a member of a cDNA library operably linked to a promoter which expresses said cDNA in said cell;
(c) exposing such cell to a DNA-damaging agent and determining the extent to which the expression of said cDNA modulates the degradation of said cyclin D1; and optionally
(d) isolating said cDNA.

4. An assay for a modulator of cell cycle control, which assay comprises:
(a) providing a cyclin D1, the APC or a component thereof which interacts with cyclin D1, together with a potential modulator compound; and
(b) determining the extent to which the presence of the potential modulator compound inhibits the interaction of said cyclin D1 and APC or component thereof.

5. An assay according to claim 4 wherein the component of the APC which interacts with cyclin D1 is a Cdc20.

6. A compound obtained using the method of claim 4 or 5, said compound being an inhibitor to the interaction of cyclin D1 with the APC.

7. A modulator of cell cycle control obtained by the method of claim 1 or 2.


SEQUENCE LISTING (7 sequences)

SEQ ID NO: 1 - protein, 13 aa, Homo sapiens
RVLRAMLKAEETC

SEQ ID NO: 2 - protein, 13 aa, Homo sapiens
RVLQNLLTIEERY

SEQ ID NO: 3 - protein, 13 aa, Homo sapiens
RVLQSLLRLEERY

SEQ ID NO: 4 - protein, 13 aa, Mus sp.
RVLRAMLKTEETC

SEQ ID NO: 5 - protein, 12 aa, Saccharomyces cerevisiae
KERQKLWLLECQ

SEQ ID NO: 6 - protein, 12 aa, Homo sapiens
RTRAALAVLKSG

SEQ ID NO: 7 - protein, 12 aa, Homo sapiens
RPRTALGDIGNK